(19) United States
(12) Patent Application Publication
(10) Pub. No.: US 2023/0330352 A1
(43) Pub. Date: Oct. 19, 2023

(54) NEEDLE COVER MANAGEMENT AND NEEDLE HIDING DEVICES FOR INJECTION SYSTEMS AND METHODS

(71) Applicant: CREDENCE MEDSYSTEMS, INC., Menlo Park, CA (US)

(72) Inventors: Mina M. Leung, Mountain View, CA (US); Stephen H. Diaz, Palo Alto, CA (US); Jeff Tillack, Foster City, CA (US)

(73) Assignee: CREDENCE MEDSYSTEMS, INC., Menlo Park, CA (US)

(21) Appl. No.: 18/300,125

(22) Filed: Apr. 13, 2023

Related U.S. Application Data
(60) Provisional application No. 63/330,415, filed on Apr. 13, 2022.

(51) Int. Cl. A61M 5/32; A61M 5/315

(52) U.S. Cl. CPC A61M 5/3243; A61M 5/31515; A61M 5/3293

(57) ABSTRACT

A system for injection includes a syringe body having proximal and distal ends and defining a syringe interior. The system also includes a stopper member disposed in the syringe interior. The system further includes a plunger member coupled to the stopper member and configured to be manipulated to insert the stopper member distally in the syringe interior relative to the syringe body. Moreover, the system includes a needle hub assembly coupled to the syringe body at the distal end thereof and including a needle hub, and a needle coupled to the needle hub. In addition, the system includes a needle shield removably coupled to the needle hub over the needle. The system also includes a telescoping sleeve coupled to the distal end of the syringe body at least partially over the needle shield.

The present application claims priority to U.S. Provisional Patent Application Ser. No. 63/330,415, filed on Apr. 13, 2022 under attorney docket number CM.30035.00 and entitled “NEEDLE COVER MANAGEMENT AND NEEDLE HIDING DEVICES FOR INJECTION SYSTEMS AND METHODS.” This application includes subject matter similar to the subject matter described in the following co-owned U.S. patent applications: (1) U.S. patent application Ser. No. 14/321,706, filed Jul. 1, 2014 and issued as U.S. Pat. No. 9,814,842 on Nov. 14, 2017 under attorney docket number CM.20001.00 and entitled “SAFETY SYRINGE”; (2) U.S. patent application Ser. No. 14/543,787, filed Nov. 17, 2014 and issued as U.S. Pat. No. 10,300,217 on May 28, 2019 under attorney docket number CM.20002.00 and entitled “SYSTEM AND METHOD FOR DRUG DELIVERY WITH A SAFETY SYRINGE”; (3) U.S. patent application Ser. No. 14/696,342, filed Apr. 24, 2015, and issued as U.S. Pat. No. 10,010,677 on Jul. 7, 2018 under attorney docket number CM.20003.00 and entitled “SYSTEM AND METHOD FOR SAFETY SYRINGE”; (4) U.S. patent application Ser. No. 15/801,239, filed on Nov. 1, 2017 and issued as U.S. Pat. No. 10,926,038 on Feb. 23, 2021 under attorney docket number CM.20011.00 and entitled “SYSTEM AND METHOD FOR SAFETY SYRINGE”; (5) U.S. patent application Ser. No. 15/801,259, filed on Nov. 1, 2017, and issued as U.S. Pat. No. 10,864,330 on Dec. 15, 2020 under attorney docket number CM.20012.00and entitled “SYSTEM AND METHOD FOR SAFETY SYRINGE”; (6) U.S. patent application Ser. No. 15/801,281 filed on Nov. 1, 2017 and issued as U.S. Pat. No. 10,912,894 on Feb. 9, 2021 under attorney docket number CM.20013.00 and entitled “CARTRIDGE SAFETY INJECTION SYSTEM AND METHODS”; (7) U.S. patent application Ser. No. 15/801,304 filed on Nov. 1, 2017 and issued as U.S. Pat. No. 10,960,144 on Mar. 30, 2021 under attorney docket number CM.20015.00 and entitled “SYSTEM AND METHOD FOR SAFETY SYRINGE”; (8) U.S. patent application Ser. No. 16/435,429 filed on Jun. 7, 2019 under attorney docket number CM.20019.00 and entitled “SYSTEM AND METHOD FOR SAFETY SYRINGE”; (9) U.S. patent application Ser. No. 16/798,188, filed on Feb. 21, 2020 under attorney docket number CM.20023.00 and entitled “SYSTEM AND METHOD FOR SAFETY SYRINGE”; (10) U.S. patent application Ser. No. 16/837,835, filed Apr. 1, 2020 under attorney docket number CM.20025.00 and entitled “POLYMERIC INJECTION SYSTEMS”; (11) U.S. patent application Ser. No. 16/908,531 filed on Jun. 22, 2020 under attorney docket number CM.20026.00 and entitled “INJECTION SYSTEM AND METHOD”; (12) U.S. patent application Ser. No. 17/031,108 filed on Sep. 24, 2020 under attorney docket number CM.20027.00 and entitled “SYSTEM AND METHOD FOR SAFETY SYRINGE”; (13) U.S. patent application Ser. No. 17/364,546 filed on Jun. 30, 2021 under attorney docket number CM.20028.00 and entitled “SYSTEM AND METHOD FOR SAFETY SYRINGE”; (14) U.S. Provisional Patent Application Ser. No. 63/094,313 filed on Oct. 20, 2020 under attorney docket number CM.30030.00 and entitled “RETRACTION MECHANISM FOR SAFE INJECTION SYSTEM”; (15) U.S. Provisional Patent Application Ser. No. 63/300,394, filed on Jan. 18, 2022 under attorney docket number CM.30034.00 and entitled “INJECTION SYSTEM AND METHOD”. The contents of the applications and patents identified herein are fully incorporated herein by reference as though set forth in full.

### FIELD OF THE DISCLOSURE

The present disclosure relates generally to injection systems, devices, and processes for facilitating various levels of control over injection processes, and more particularly to devices and methods related to handling needle covers four injection systems, with or without safety features distinct from the needle cover handling devices, in healthcare environments.

### BACKGROUND

Millions of syringes, such as that depicted in FIG. 1A (2), are consumed in healthcare environments every day. A typical syringe (2) comprises a tubular body (4), a plunger (6), and an injection needle (8). As shown in FIG. 1B, such a syringe (2) may be utilized not only to inject fluid into a patient, but also to withdraw or expel fluid out of or into a container such as a medicine bottle, vial, bag, or other drug containment system (10). Indeed, due to regulatory constraints in some countries such as the United States as well as sterility maintenance concerns, upon use of a medicine bottle (10) with a syringe (2) as shown in a particular patient's environment, such medicine bottle may only be utilized with a single patient and then must be disposed of—causing significant medical waste from bottle and remaining medicine disposal, and even contributing to periodic shortages of certain critical drugs. Referring to FIG. 2A, three Luer-type syringes (12) are depicted, each having a Luer fitting geometry (14) disposed distally, so that they may be coupled with other devices having similar mating geometry, such as the Luer manifold assembly (16) depicted in FIG. 2B. The Luer manifold assembly of FIG. 2B may be used to administer liquid drugs to the patient intravenously with or without the use of an intravenous infusion bag. The Luer fittings (14) of the syringes of FIG. 2A may be termed the “male” Luer fittings, while those of FIG. 2B (18) may be termed the “female” Luer fittings; one of the Luer interfaces may be threaded (in which case the configuration may be referred to as a “Luer lock” configuration) so that the two sides may be coupled by relative rotation, which may be combined with compressive loading. In other words, in one Luer lock embodiment, rotation, possibly along with compression, may be utilized to engage threads within the male fitting (14) which are configured to engage a flange on the female fitting (18) and bring the devices together into a fluid-sealed coupling. In another embodiment, tapered interfacing geometries may be utilized to provide for a Luer engagement using compression without threads or rotation (such a configuration may be referred to as a “slip-on” or “conical” Luer configuration). While such Luer couplings are perceived to be relatively safe for operators, there is risk of medicine spilling/leaking and parts breakage during assembly of a Luer coupling. The use of needle injection configurations, on the other hand, carries with it the risk of a sharp needle contacting or stabbing a person or structure that is not desired. For this reason, so called “safety syringes” have been developed.

One embodiment of a safety syringe (20) is shown in FIG. 3, wherein a tubular shield member (22) is spring biased to cover the needle (8) when released from a proximal/retracted position relative to the syringe body (4). The tubular needle shield (22) is “locked” in the distal/extended configuration, such that the needle shield (22) can no longer be returned to the proximal/retracted position, to prevent accidental needle sticks after injection.

Another embodiment of a safety syringe (24) is shown in FIGS. 4A-4B. With such a configuration, after full insertion of the plunger (6) relative to the syringe body (4), the retractable needle (26) is configured to retract (28, 26) back to a safe position within the tubular body (4), as shown in FIG. 4B. Such a configuration which is configured to collapse upon itself may be associated with blood spatter/aerosolization problems, the safe storage of pre-loaded energy which may possibly malfunction and activate before desirable, loss of accuracy in giving full-dose injections due to residual dead space within the spring compression volume, and/or loss of retraction velocity control which may be associated with pain and patient anxiety.

Further complicating the syringe marketplace is an increasing demand for prefilled syringe assemblies such as those depicted in FIGS. 5A and 5B, which generally comprise a syringe body, or “drug enclosure containment delivery system”, (34), a plunger tip, plug, or stopper (36), and a distal seal or cap (35) which may be fitted over a Luer type interface (FIG. 5A shows the cap 35 in place; FIG. 5B has the cap removed to illustrate the Luer interface 14). Liquid medicine may reside in the volume, or medicine reservoir, (40) between the distal seal and the distal end (37) of the plunger tip (36). The plunger tip (36) may comprise a standard butyl rubber material and may be coated, such as with a biocompatible lubricious coating (e.g., polytetrafluoroethylene (“PTFE”)), to facilitate preferred sealing and relative motion characteristics against the associated syringe body structure and material. The proximal end of the syringe body (34) in FIG. 5B comprises a conventional integral syringe flange (38), which is formed integral to the material of the syringe body (34). The flange (38) is configured to extend radially from the syringe body (34) and may be configured to be a full circumference, or a partial circumference around the syringe body (34). A partial flange is known as a “clipped flange” while the other is known as a “full flange.” The flange is used to grasp the syringe with the fingers to provide support for pushing on the plunger to give the injection. The syringe body (34) preferably comprises a translucent material such as a glass or polymer. To form a contained volume within the chamber or reservoir (40), and to assist with expulsion of the associated fluid through the needle, a plunger tip (36) may be positioned within the syringe body (34). The syringe body (34) may define a substantially cylindrical shape (i.e., so that a plunger tip 36 having a circular cross-sectional shape may establish a seal against the syringe body (34)), or be configured to have other cross-sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and anti-needle-stick. Thus, certain suppliers have moved to more “vertical” solutions, such as that (41) featured in FIG. 5C, which attempts to meet all of the standards, or at least a portion thereof, with one solution; as a result of trying to meet these standards for many different scenarios, such products may have significant limitations (including some of those described above in reference to FIGS. 3-4B) and relatively high inventory and utilization expenses.

Most injection systems include a needle cover (e.g., rigid needle shield, needle shield, etc.) removably coupled to a needle hub and configured to cover the sharp distal end of the needle to minimize accidental needle sticks, to protect the needle from mechanical damage, and to seal the injection system before use. Existing injection systems are configured such that needle covers are removed manually by a user's hands. Needle covers are secured to needle hubs. Significant force must be exerted by the user's hands to remove needle covers from needle hubs. The user's hands are proximate the sharp distal end of the needle when removing the needle cover. As a result of this proximity, the removal of the needle cover can result in accidental needle sticks (e.g., with a bounce back of the hand grasping the needle cover or movement of the hand holding the injection system). When a user applies force to the proximal end of the needle cover to avoid needle sticks, the needle cover may be launched from the needle hub and end up as unwanted trash on the floor of the medical facility. Accordingly, there exists a need for needle cover handling devices for a more controlled and safer removal of needle covers from injection systems.

Further, for multiple chamber injection systems such as those described in U.S. patent application Ser. No. 15/801,259, which was previously incorporated by reference herein, pressure may build up in a distal chamber when mixing drug components therein prior to injection with the distal end of the needle sealed by a needle cover. Accordingly, there exists a need for needle handling devices to removably hold a needle cover apart from but still covering the sharp distal end of the needle to allow venting of multiple chamber injection systems. In particular, there is a need for needle cover handling devices, which may utilize the existing and relatively well-controlled supply chain of conventionally delivered syringe assemblies.

### SUMMARY

Embodiments are directed to injection systems. In particular, the embodiments are directed to needle cover handling devices for injection systems.

In one embodiment, a system for injection includes a syringe body having proximal and distal ends and defining a syringe interior. The system also includes a stopper member disposed in the syringe interior. The system further includes a plunger member coupled to the stopper member and configured to be manipulated to insert the stopper member distally in the syringe interior relative to the syringe body. Moreover, the system includes a needle hub assembly coupled to the syringe body at the distal end thereof and including a needle hub, and a needle coupled to the needle hub. In addition, the system includes a needle shield removably coupled to the needle hub over the needle. The system also includes a telescoping sleeve coupled to the distal end of the syringe body at least partially over the needle shield.

In one or more embodiments, the needle hub defines a circumferential groove, and the needle shield includes an elastic material configured to interfere with the circumferential groove in the needle hub to resist removal of the needle shield from the needle hub.

In one or more embodiments, the telescoping sleeve includes fixed and movable members configured to telescope relative to each other along a longitudinal axis of the telescoping sleeve. The movable member may be disposed at least partially radially outside of the fixed member. The movable member may define an annular space, and the fixed member may be at least partially disposed in the annular space. The telescoping sleeve may include a spring disposed in the annular space radially inward of the fixed member. The spring may reduce a force required to remove the needle shield from the needle hub. The spring may not exert a resistive force noticeable to a user performing an injection using the system.

In one or more embodiments, the movable member defines a plurality of inwardly extending members configured to interfere with a proximal end of the needle shield when the needle shield is coupled to the needle hub. The plurality of inwardly extending members may be configured to exert a distally directed force from the movable member to the proximal end of the needle shield when the needle shield is coupled to the needle hub. The fixed member may define an outwardly extending flange at a distal end thereof, the movable member may define an inwardly extending flange at a proximal end thereof, and the outwardly extending flange and the inwardly extending flange may be configured to interfere with each other to limit distal movement of the movable member relative to the fixed member. The movable member may define an outwardly sloping surface configured to facilitate application of a distally directed force to the movable member of the telescoping sleeve.

In one or more embodiments, the telescoping sleeve has a retracted configuration and an extended configuration. The fixed and movable members may substantially overlap each other when the telescoping sleeve is in the retracted configuration. The movable member may be moved distally relative to the fixed member with minimal overlap between the fixed and movable members when the telescoping sleeve is in the extended configuration. The telescoping sleeve may also have a fully retracted configuration. The fixed member may define a side opening, and the movable member may include a latch configured to enter the opening when the telescoping sleeve is in the fully retracted configuration, thereby preventing the movable member from moving distally relative to the fixed member.

In one or more embodiments, a distal end of the movable member is substantially aligned with a distal tip of the needle along a longitudinal axis of the system when the telescoping sleeve is in the extended configuration. A distal end of the movable member may prevent a distal tip of the needle from being seen from proximal of the movable member when the telescoping sleeve is in the extended configuration. A distal end of the movable member may be configured to contact a patient's skin at substantially the same time as a distal tip of the needle during an injection using the system when the telescoping sleeve is in the extended configuration.

In one or more embodiments, the system also includes a retaining clip configured to secure the telescoping sleeve to the needle hub. The movable member may define a plurality of distally extending members configured to removably retain the needle shield away from a distal end of the needle after the needle shield is removed from the needle hub. Each of the plurality of distally extending members may define an inwardly extending rib configured to contact the needle shield after the needle shield is removed from the needle hub.

In one or more embodiments, the movable member defines a substantially flat distally facing surface. The system may also include a safe rejection system, such as that described herein and shown in FIGS. 6A to 7P, configured to retract the needle at least partially within the syringe body after an injection with the system is completed such that a distal end of the needle is disposed within the needle hub or the syringe body.

In one or more embodiments, the movable member defines a pair of inwardly extending latches configured to interfere with a proximal end of the needle shield when the needle shield is coupled to the needle hub. The pair of inwardly extending latches may be configured to exert a distally directed force from the movable member to the proximal end of the needle shield when the needle shield is coupled to the needle hub.

In one or more embodiments, the telescoping sleeve has a retracted configuration and an extended configuration. The movable member may substantially cover the fixed member when the telescoping sleeve is in the retracted configuration. The movable member may be moved distally relative to the fixed member when the telescoping sleeve is in the extended configuration.

In one or more embodiments, the fixed member defines a pair of slots. The pair of inwardly extending latches may be partially disposed in the pair of slots when the telescoping member is in the retracted or extended configurations. The pair of inwardly extending latches may be configured to interfere with respective proximally facing surfaces at the distal ends of the pair of slots to limit distal movement of the movable member relative to the fixed member.

In one or more embodiments, the fixed member defines a plurality of inwardly extending skids configured to contact an inner surface of the movable member to retain the telescoping member in the extended configuration. The fixed member may define an inwardly extending rib configured to contact the needle shield after the needle shield is removed from the needle hub.

In one or more embodiments the telescoping sleeve also has a fully retracted configuration. The pair of inwardly extending latches may also be configured to interfere with a proximal surface of the fixed member when the telescoping sleeve is in the fully retracted configuration, thereby preventing the movable member from moving distally relative to the fixed member. A distal end of the movable member may be substantially aligned with a distal tip of the needle along a longitudinal axis of the system when the telescoping sleeve is in the retracted configuration.

The aforementioned and other embodiments of the invention are described in the Detailed Description which follows.

### BRIEF DESCRIPTION OF THE DRAWINGS

FIGS. 1A to 5C illustrate various aspects of conventional injection syringe configurations.

FIGS. 6A and 6B are perspective and longitudinal cross-section views illustrating various aspects of syringe based dual chamber safe injection systems with which the needle cover handling devices described herein may be used and wherein a distal needle end/tip may be withdrawn into a protected configuration after use according to some embodiments.

FIGS. 7A to 7P are side and longitudinal cross-section views illustrating various aspects of syringe based dual chamber safe injection systems with which the needle cover handling devices described herein may be used and during steps in methods for mixing and injecting using same according to some embodiments.

FIGS. 8 and 9 are perspective and side exploded views of a needle cover handling device/telescoping member for use with an injection system according to some embodiments.

FIG. 10 is a perspective partially exploded view of a needle cover handling device/telescoping member for use with an injection system according to some embodiments.

FIGS. 11 and 12 are longitudinal cross-sectional and side views of a needle cover handling device/telescoping member mounted on an injection system according to some embodiments.

FIGS. 13 and 14 are longitudinal cross-sectional views of a needle cover handling device/telescoping member mounted on an injection system in respective retracted and extended configurations according to some embodiments.

FIG. 15 is a partial cut-away perspective view of a needle cover handling device/telescoping member for use with an injection system according to some embodiments.

FIGS. 16A and 16B are longitudinal cross-sectional and perspective views of a needle cover handling device/telescoping member mounted on an injection system in an extended configuration according to some embodiments.

FIGS. 17A and 17B are longitudinal cross-sectional and perspective views of a needle cover handling device/telescoping member mounted on an injection system in a fully retracted configuration according to some embodiments.

FIG. 18 is a perspective exploded view of a needle cover handling device/telescoping member for use with an injection system according to some embodiments.

FIG. 19 is a perspective partially exploded view of a needle cover handling device/telescoping member for use with an injection system according to some embodiments.

FIGS. 20, 21, and 22 are detailed side, longitudinal cross-sectional, and side views of a needle cover handling device/telescoping member mounted on an injection system according to some embodiments.

FIG. 23 is a longitudinal cross-sectional view of a needle cover handling device/telescoping member mounted on an injection system in a retracted configuration according to some embodiments.

FIG. 24 is a longitudinal cross-sectional view of a needle cover handling device/telescoping member mounted on an injection system in an extended configuration according to some embodiments.

FIG. 25 is a partial cut-away perspective view of a needle cover handling device/telescoping member for use with an injection system according to some embodiments.

FIGS. 26A, 26B, and 28 are longitudinal cross-sectional, perspective, and perspective views of a needle cover handling device/telescoping member mounted on an injection system in an extended configuration according to some embodiments.

FIG. 27 is a partial cut-away perspective view of a needle cover handling device/telescoping member according to some embodiments.

FIGS. 29A, 29B, and 30 are longitudinal cross-sectional, perspective, and longitudinal partial cross-sectional views of a needle cover handling device/telescoping member mounted on an injection system in a fully retracted configuration according to some embodiments.

FIG. 31 is a perspective exploded view of a needle cover handling device/telescoping member for use with an injection system according to some embodiments.

FIG. 32 is a side view of a needle cover handling device/telescoping member mounted on an injection system according to some embodiments.

FIGS. 33 and 34 are longitudinal cross-sectional views of a needle cover handling device/telescoping member mounted on an injection system in respective retracted and extended configurations according to some embodiments.

FIGS. 35A, 35B, and 35C are longitudinal cross-sectional views of a needle cover handling device/telescoping member mounted on an injection system in respective short, medium, and long extended configurations according to some embodiments.

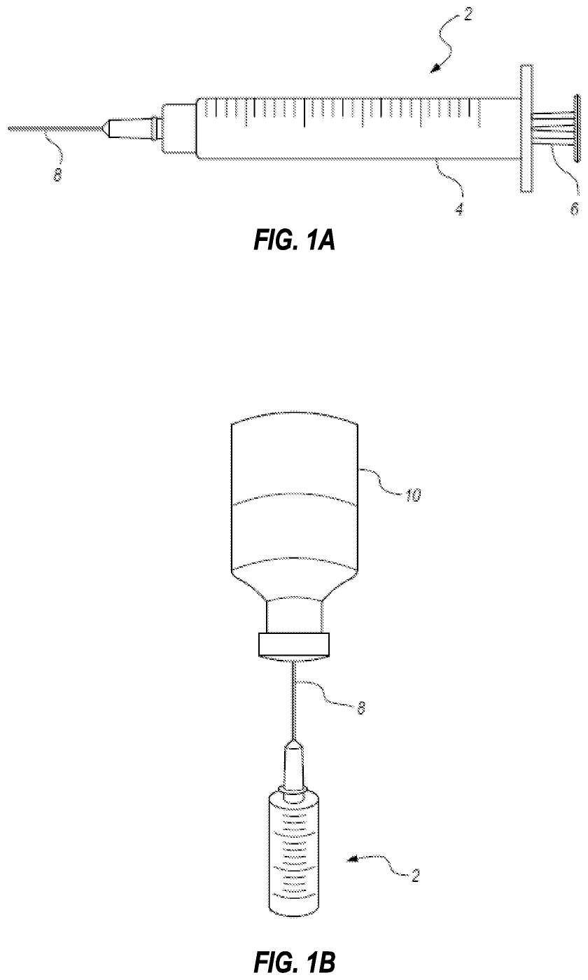

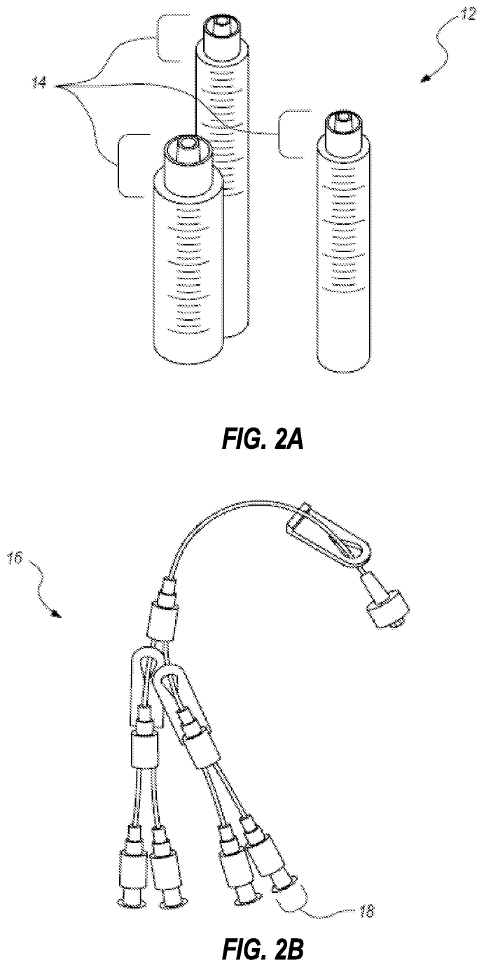

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS Exemplary Dual Chamber Safe Syringe Systems With Which Needle Cover

Handling Devices May Be Used

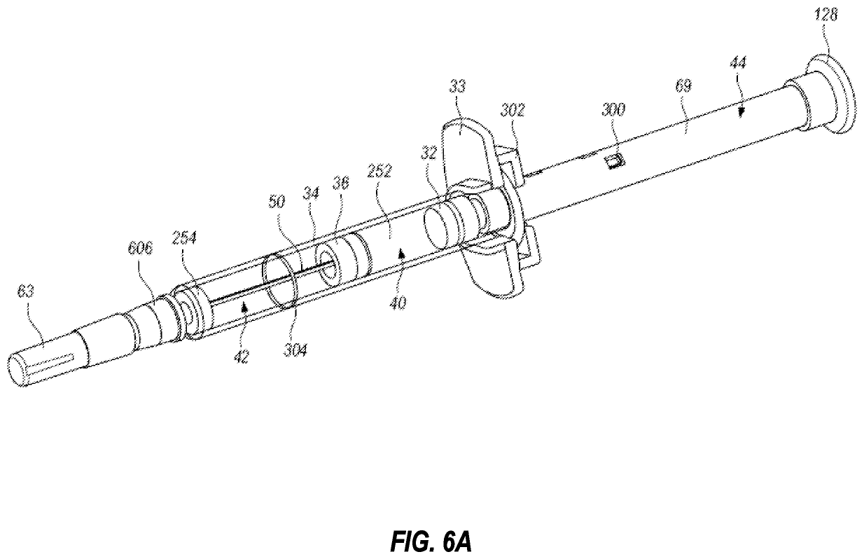

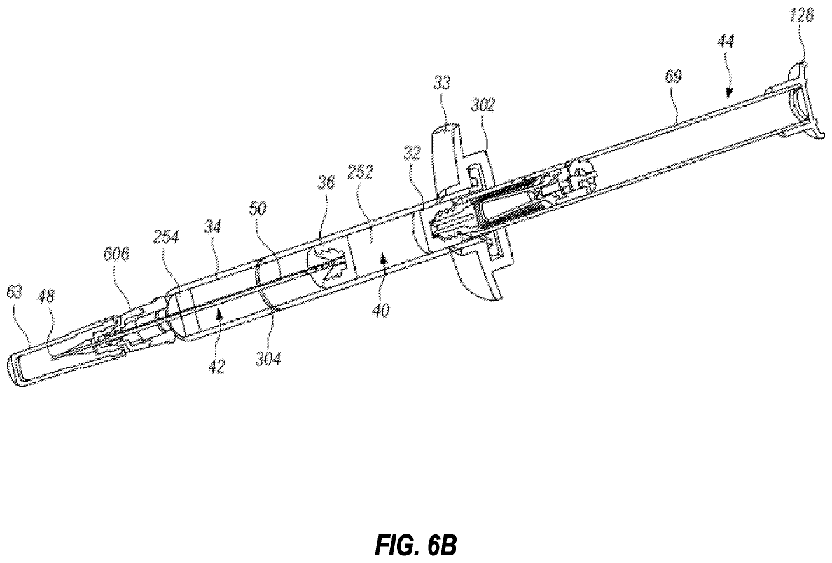

Referring to FIGS. 6A-6B, a perspective and a longitudinal cross section view of a dual chamber safe injection system are shown, with a conventional off-the-shelf pre-filled syringe body (34) with conventional proximal and distal stopper members (32, 36) disposed therein. The proximal and distal stopper members (32, 36) together with the syringe body (34) define proximal and distal medicine chambers (40, 42). The proximal and distal stopper members (32, 36) occlude the proximal and distal ends of the proximal medicine chamber (40). The distal stopper member (36) occludes a proximal end of the distal medicine chamber (42). A needle hub assembly (606) is disposed at the distal end of the distal medicine chamber (42) with a needle cover (63) installed for storage. The dual chamber safe injection system controls transfer of a first medicine component from the proximal medicine chamber (40) to the distal medicine chamber (42) and exit of a mixed/combined medicine from the distal medicine chamber (42) distally subject to sequential insertion of a plunger member (44) relative to the syringe body (34) to various degrees by a user. The plunger member (44) includes the proximal stopper member (32), a plunger housing member (69) and a plunger manipulation interface (128). The first medicine component (252) located in the proximal medicine chamber (40) may be a liquid such as aqueous or oil based medicine solutions, a gel, or the first medicine component may be a diluent for mixing with the second medicine component (254) in the distal medicine chamber (42). The second medicine component (254) in the distal medicine chamber (42) may be a dry form medicine such as a powder, microspheres, emulsion, lyophilized or freeze dried medicine, or a cake like solid medicine.

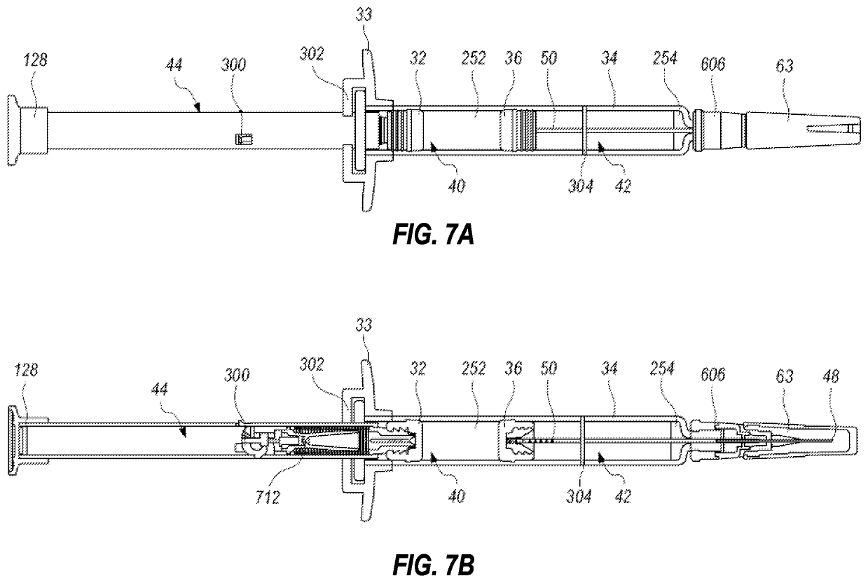

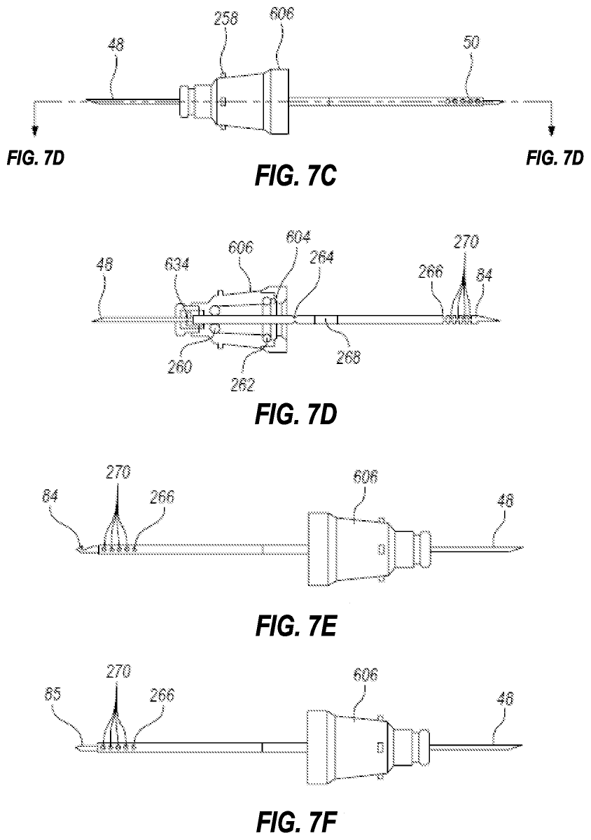

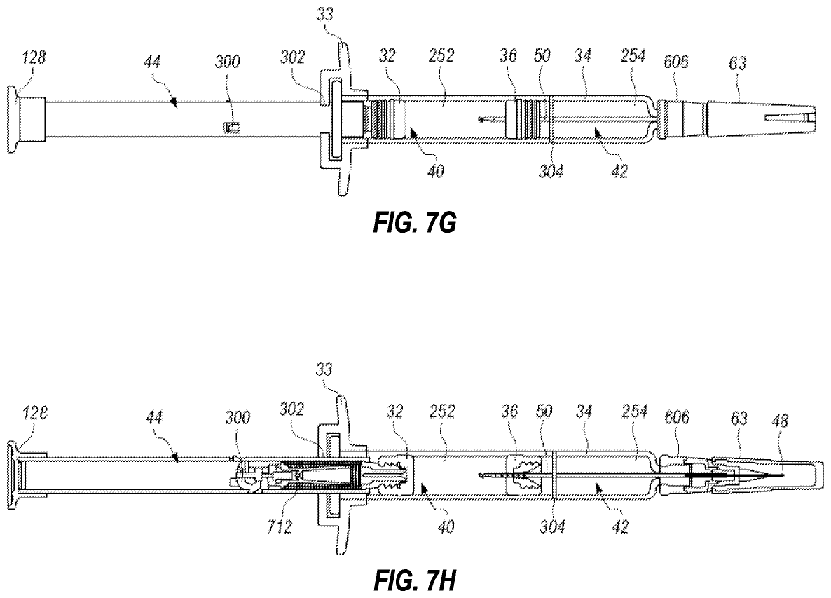

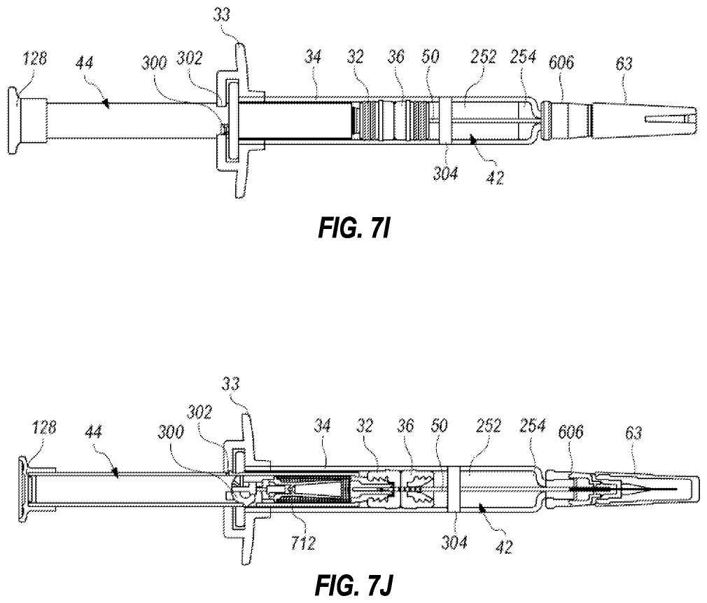

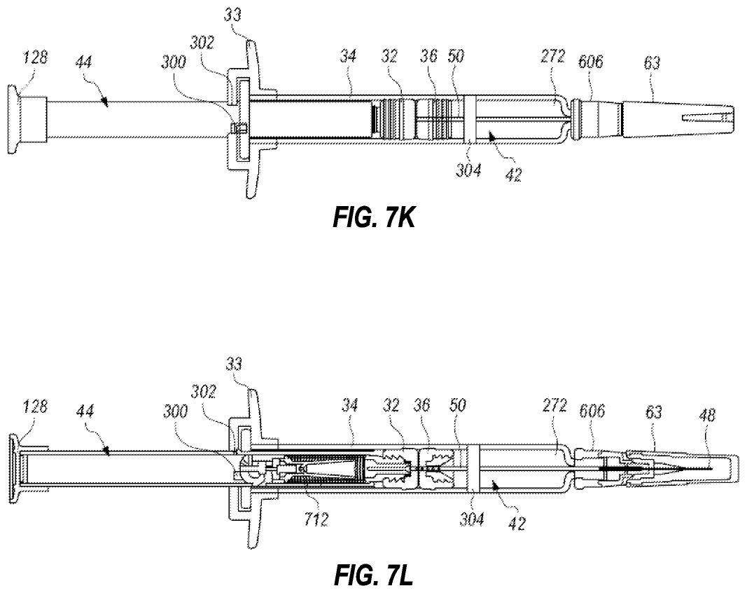

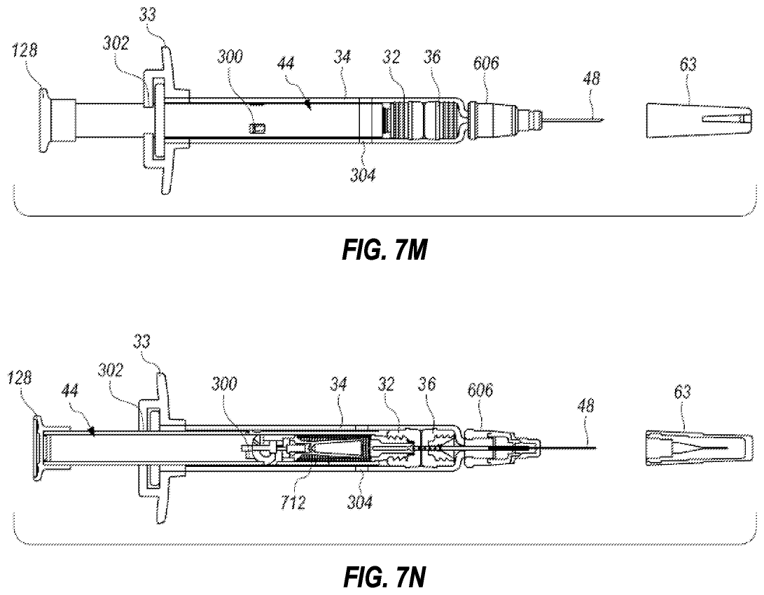

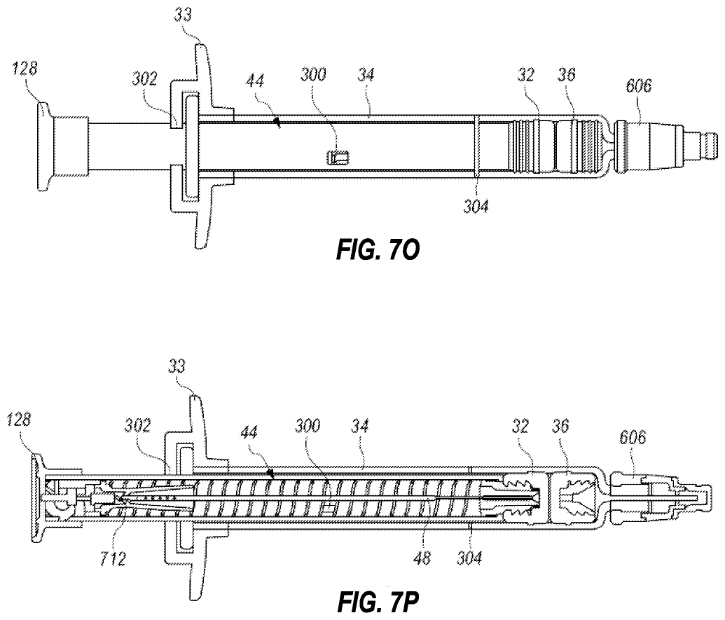

The dual chamber safe injection system has a staked needle configuration wherein upon presentation to the user, a needle assembly, comprising a needle hub assembly (606), a needle distal end/tip (48), a needle joining member, and a needle proximal end (50) are mounted in position ready for injection after removal of a needle cover (63) which may comprise an elastomeric sealing material on its internal surface to interface with the needle distal end (48) or the distal housing portion (610) during storage. Alternatively, the needle cover (63) may comprise a vent (not shown) for allowing pressure resulting from the transfer and mixing of the medicine components to escape from inside the syringe body (34) while preventing contamination from entering the syringe body (34). While, the staked needle is depicted as mounted in position, the staked needle may be removably coupled to the syringe body (34) using a Luer interface (not shown), with the proximal end (50) of the needle member extending through the Luer interface and into the distal medicine chamber (42). In the embodiments depicted in FIGS. 6A-7P, a significant portion of the safe needle retraction hardware resides within a plunger housing (69).

The dual chamber safe injection system has a staked needle configuration wherein upon presentation to the user, a needle assembly, including a needle spine assembly (“needle”) (76) and a needle hub assembly (606) are mounted in position ready for injection after removal of a needle cover (63) which may comprise an elastomeric sealing material on its internal surface to interface with a needle distal end (78) and/or a distal housing portion during storage. Alternatively, the needle cover (63) may comprise a vent (not shown) for allowing pressure resulting from the transfer of the first medicine component/diluent (252) to escape from inside the syringe body (34) while preventing contamination from entering the syringe body (34). While, the staked needle is depicted as mounted in position, the staked needle may be removably coupled to the syringe body (34) using a Luer slip or a Luer lock interface (not shown), with the proximal end (50) of the needle member extending through the Luer interface and into the distal chamber (42). Alternatively, the needle may be fixedly or removably mounted to the flange on a cartridge body instead of a syringe. Such cartridge injection systems are disclosed in U.S. patent application Ser. No. 15/801,281, which was previously incorporated by reference herein. In the embodiments depicted in FIGS. 6A and 6B, a significant portion of the safe needle retraction hardware resides within a plunger housing (69).

Referring to FIGS. 7A-7P, various aspects of configurations designed to facilitate injection of multi-part medications and retractions of a needle into a syringe body are illustrated, wherein two or more medication components are combined to form an injection combination or solution shortly before delivery into the patient. In one variation, a liquid first medicine component/diluent (252) may be combined with a substantially non-liquid second medicine component (254), such as a powdered form, of a drug agent, such as a freeze-dried or lyophilized drug component, shortly before injection. The configurations described herein in reference to FIGS. 7A-7P relate to dual-chamber configurations, wherein two or more chambers within the same syringe body (34) are utilized to carry, mix, and inject an injection solution.

Referring to FIG. 7A and 7B, proximal and distal medicine chambers (40, 42) are formed by a distal stopper member (36) in between two portions of the interior of a syringe body (34), such that the distal medicine chamber (42) contains an air or gas gap, as well as a non-liquid medication (254); a proximal medicine chamber (40), on the opposite side of the distal stopper member (36) contains a liquid diluent (252), which is proximally contained by a proximal stopper member (32). The liquid diluent (252) is a first component of a medicine and the non-liquid medication (254) is a second component of the medicine.

Referring to FIG. 7C, and the associated cross sectional view in FIG. 7D, various components of a needle hub assembly (here a so-called “staked” needle hub assembly (606) is illustrated, but other needle assemblies as described below, including Luer-coupled as well as staked configurations, may be utilized). Lug features (258) are configured to assist with coupling the needle hub assembly (606) to a needle cover (63), as shown in FIG. 7A, for example. A small O-ring may be utilized as a sealing member (260) around the needle shaft, while a larger O-ring may be utilized as a sealing member (262) at the syringe body (34)/needle hub assembly (606) interface. Alternatively, the small O-ring (260) and the large O-ring (262) may be combined into a single seal that performs both of the O-ring sealing functions. Also, the small O-ring (260) may be used to seal both around the needle shaft and to the syringe body (34).

The needle includes a plurality (e.g., four) of proximal openings/ports (270) configured to allow for entry of a liquid diluent, to be expelled out of a more distally-located middle opening/aperture (266); a lumen plug (268) occludes the needle lumen to create the flow path from the proximal openings (270) to the middle opening (266) under conditions such as those described above in reference to FIGS. 6N and 7H. The needle also includes a distal opening (264) on the opposite side of the lumen plug (268) from the middle opening (266). The distal opening (264) is fluidly coupled to the needle distal end (48) through the needle to inject liquid into a patient.

Referring to FIG. 7E, a proximal harpoon interface (84) is configured to serially penetrate proximal and distal stopper members (32, 36), and couple with a coupling feature (such as a needle retention feature are illustrated, for example, in FIGS. 7N and 7P, element (712)) in the plunger member (44). FIG. 7F illustrates a spike style harpoon coupling interface (85) that is configured to serially pierce both proximal and distal stopper members (32, 36) and couple with a coupling feature in the plunger member (44) to retract the needle member at least partially into the plunger member (44) after the injection has been given to the patient.

FIGS. 7A, 7B, and 7G-7P illustrate a sequence of actions for an injection procedure utilizing a dual chamber safe injection system such as that described above. Referring to FIGS. 7A and 7B, an injection assembly is in a stable configuration wherein it may be shipped or brought to an injection patient care scenario; a first drug component/liquid diluent (252) is isolated from a second non-liquid drug component (254), both within a syringe body on opposite sides of a distal stopper member (36).

FIGS. 7G and 7H illustrate initial insertion movement of the plunger member (44), advancing the distal (36) and proximal (32) stopper members together relative to the syringe body (34). Referring to FIG. 7H, with advancement sufficient to stab the proximal end (50) of the needle assembly across the distal stopper member (36), a fluid pathway is formed between the two previously isolated chambers (40, 42) of the syringe body (34), such that the liquid first drug component (252) in the proximal medicine chamber (40) may flow into at least one of the proximal openings (270), through the transfer pipe (46), and exit the more distal middle opening (266), to reach the non-liquid second drug component (254) in the distal medicine chamber (42).

FIGS. 71 and 7J illustrate that with further insertion until the stopper members (36, 32) are immediately adjacent each other, the liquid first drug component/diluent (252) has moved into the distal medicine chamber (42) to join the non-liquid second drug component (254). FIGS. 7K and 7L illustrate that with time and/or manual agitation, the liquid first drug component/diluent (252) and previously non-liquid second drug component (254) become mixed to form a mixed medication solution (272).

In some embodiment, especially with lyophilized non-liquid second drug components, the mixed medication solution (272) may be formed with minimal or no agitation or time passage. In another embodiment, especially with drugs which are held in suspension or emulsified drugs, vigorous shaking may be necessary to facilitate mixing. In the case of vigorous shaking, it is useful to the user to be able to remove their thumb from the plunger manipulation interface (128). During transfer of liquid first medicine component (252) from the proximal to the distal medicine chambers (40, 42) pressure may build up in the distal medicine chamber (42). This pressure acts upon the proximal and distal stopper members (32, 36) to resist stopper motion. The pressure buildup may also move the stopper members (32, 36) and plunger manipulation interface (128) proximally if the user does not have their thumb restraining the plunger member (44). Mixed configuration latches or “mix clicks” in the plunger member (44) (described in U.S. patent application Ser. No. 15/801,259, which was previously incorporated by reference herein) may be utilized to provide resistance to plunger manipulation interface (128) motion due to pressure buildup and allow the user to release their thumb from the plunger manipulation interface (128) for shaking or mixing of the drug. The mix clicks may also provide an audible and/or tactile indication that the transfer of liquid first medicine component (252) has been completed. The distal medicine chamber (42) may also include an agitation device, which assists in mixing of the medicine components.

With the assembly ready for injection of the mixed solution (272), the needle cover (63) may be removed and the patient may be injected with the exposed needle distal end (48) with depression/insertion of the plunger member (44) and associated stopper members (36, 32) as shown in FIGS. 7M and 7N. Referring to FIGS. 70 and 7P, with full depression/insertion of the plunger member (44) and associated stopper members (32, 36), the sharp needle distal end/point (48) may automatically retract at least partially through the distal and proximal stopper members (36, 32) to a safe position within either the syringe body (34), the needle hub assembly (606), or at least partially within the plunger member (44). Automatic retraction of the needle at least partially within the plunger is described in U.S. patent application Ser. No. 14/696,342, which was previously incorporated by reference herein.

Further details regarding multiple chamber injection systems (components, methods using same, etc.) with which the needle cover handling devices described herein may be used are disclosed in U.S. patent application Ser. No. 15/801,259, which were all previously incorporated by reference herein.

### Exemplary Needle Cover Handling and Needle Hiding Devices for Use with Injection Systems

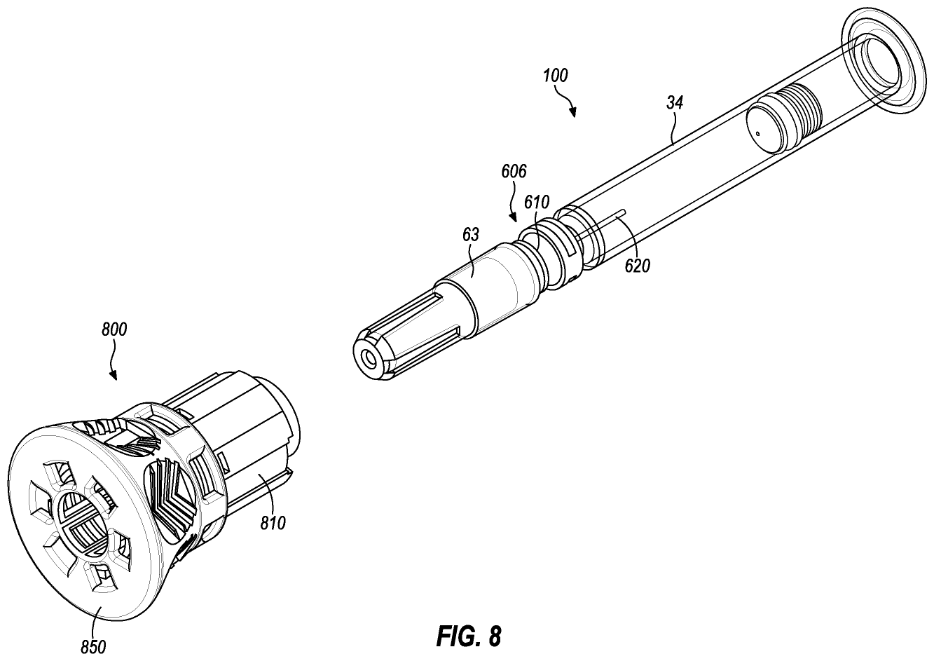

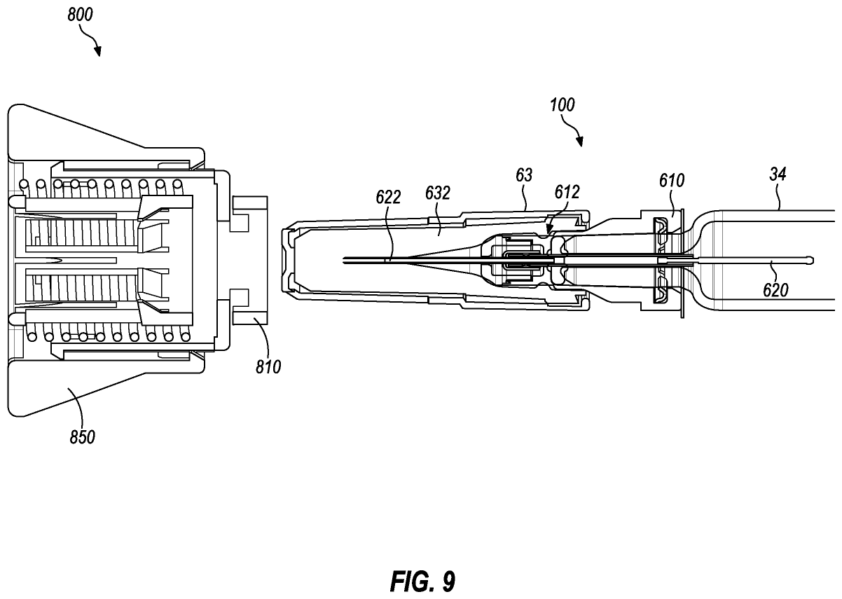

FIGS. 8 and 9 depict a needle cover handling device (800) for use with an injection system (100) before coupling the needle cover handling device (800) onto the injection system (100) according to some embodiments. While the injection system (100) is a single chamber injection system, the needle cover handling device (800) can be utilized with multiple chamber injection systems such as those depicted in FIGS. 6A to 7P without any modification.

The injection system (100) includes an injection system body (34) having a needle hub assembly (606) including a needle hub (610) and a needle (620) coupled to the needle hub (610). The injection system (100) also includes a needle cover (63) (see FIG. 9) disposed over a sharp distal end (622) of the needle (620) when the injection system (100) is in a transport/storage configuration. The needle cover (63) includes an elastic material (632) configured to interfere with a circumferential groove (612) defined by the needle hub (610) to secure the needle cover (63) onto the needle hub (610). The elastic material (632) also protects the needle (620) from mechanical damage, and seals the injection system (100) before use. The needle cover (63) minimizes accidental needle sticks during use.

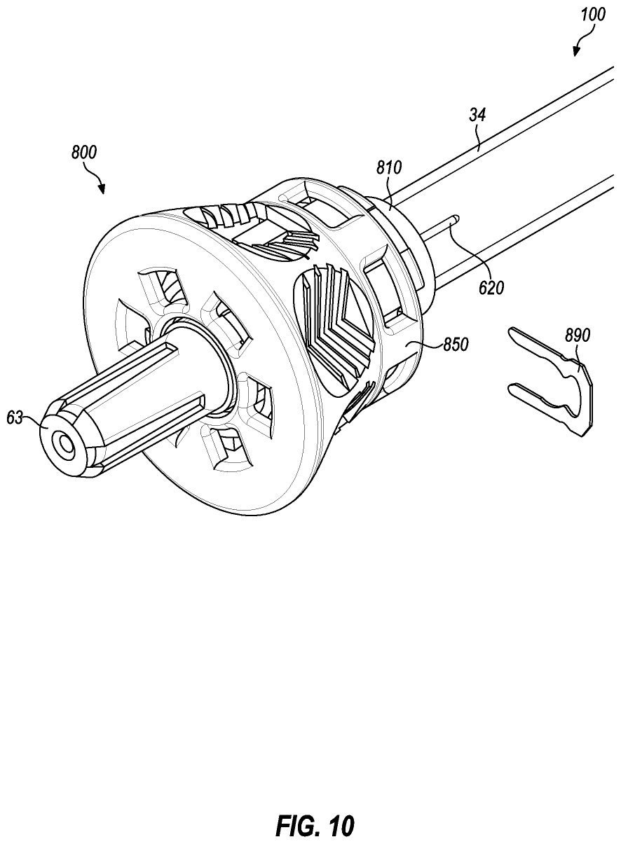

As shown in FIG. 9, the needle cover handling system (800) is a telescoping sleeve (800). The telescoping sleeve (800) includes a fixed member (810) and a movable member (850), which are configured to telescope relative to each other to change the length of the telescoping sleeve (800). During assembly of the telescoping sleeve (800) onto the injection system (100), the fixed member (810) is coupled to the needle hub (610) and the injection system body (34) with a retaining clip (890) (see FIG. 10). As seen by comparing FIGS. 9 and 10, the telescoping sleeve (800) is installed onto the injection system (100) by sliding the telescoping sleeve (800) over the needle cover (63) and securing the telescoping sleeve (800) with the retaining clip (890).

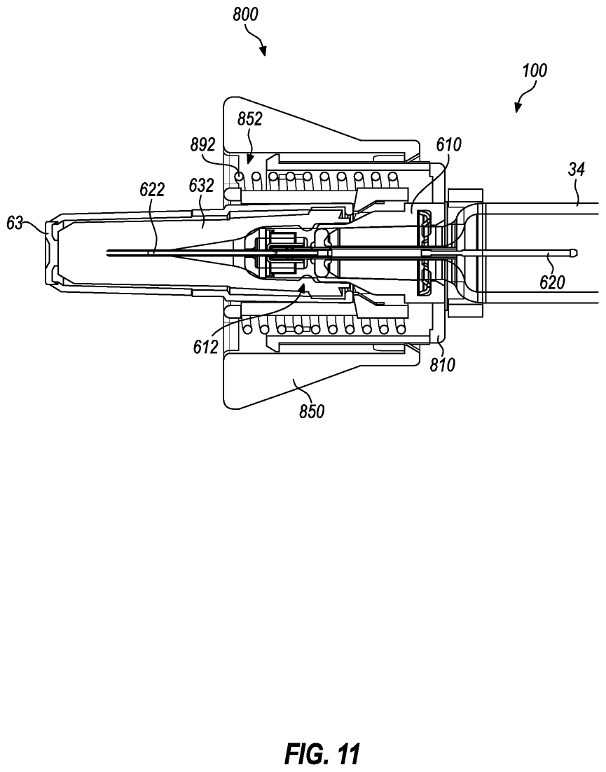

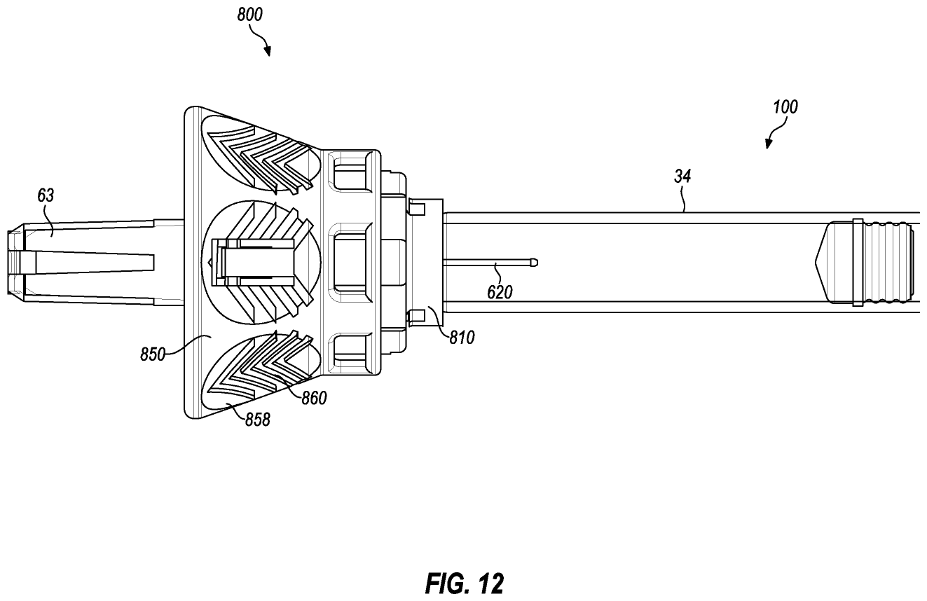

FIGS. 11 and 12 depict the telescoping sleeve (800) installed onto the injection system (100). As shown in FIG. 11, the movable member (850) defines an annular space (852) in which a distal portion of the fixed member (810) is disposed. An outer portion of the movable member (850) is disposed radially outside of the fixed member (810).

The telescoping member (800) also includes a spring (892) disposed in the annular space (852) defined by the movable member (850). The spring (892) reduces the force required to remove the needle cover from the needle hub (610). At the same time, the spring (892) does not exert a resistive force noticeable to a user performing an injection using the injection system (100).

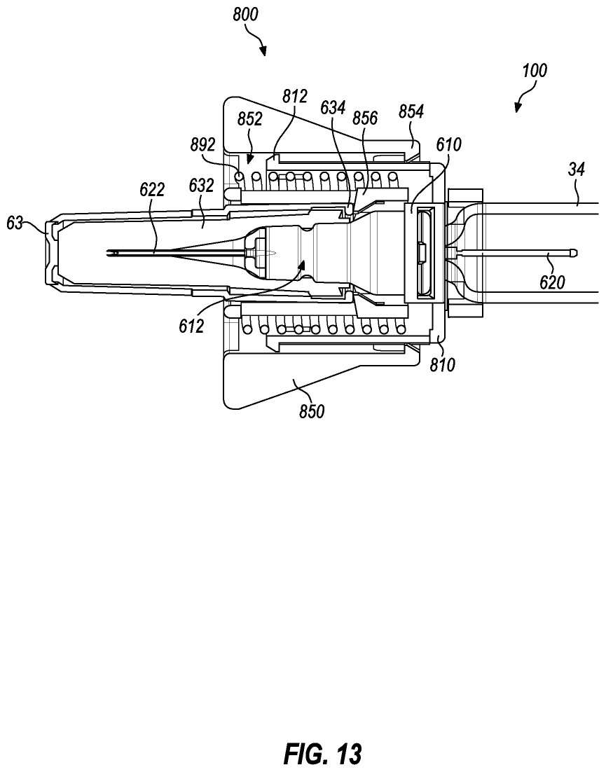

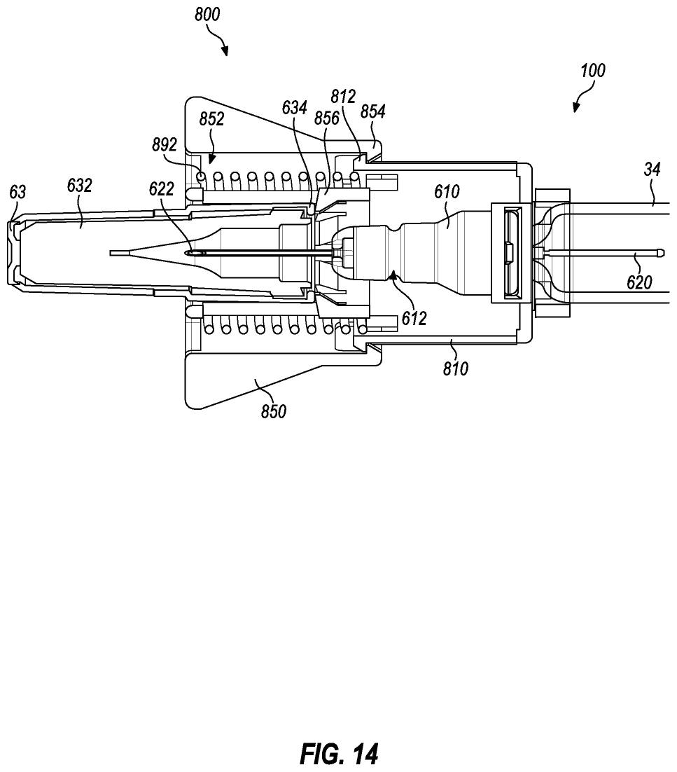

FIGS. 13 and 14 depict the telescoping member (800) in a retracted configuration (FIG. 13) and an extended configuration (FIG. 14). In the retracted configuration depicted in FIG. 13, the fixed and movable members (810, 850) substantially overlap each other. In the extended configuration depicted in FIG. 14, the movable member (850) is moved distally relative to the fixed member (810) such that there is minimal overlap between the fixed and movable members (810, 850). The telescoping member (800) is shorter in the retracted configuration compared to the extended configuration.

The fixed member (810) defines an outwardly extending flange (812) a distal end thereof, and the movable member (850) defines a corresponding inwardly extending flange (854) at a proximal end thereof. The outwardly extending flange (812) and the inwardly extending flange (854) are configured to interfere with each other to limit distal movement of the movable member (850) relative to the fixed member (810), as shown in FIG. 14.

The movable member (850) also defines a plurality of inwardly extending members (856) configured to interfere with a proximal end (634) of the needle cover (63) when the needle cover (63) is coupled to the needle hub (610). As such, the plurality of inwardly extending members (856) are configured to exert a distally directed force from the movable member (850) to the proximal end (634) of the needle cover (63), thereby removing the needle cover (63) from the needle hub (610). The distally directed force transferred from the movable member (850) through the plurality of inwardly extending members (856) is sufficient to overcome the resistive force between the elastic material (632) of the needle cover (63) and the outside surface of the needle hub (620) including the groove (612) form therein.

The movable member (850) also defines an outwardly sloping surface (858) configured to facilitate application of a distally directed force to the movable member (850) using the fingers and thumb of one hand of a user. As shown in FIG. 12, the outwardly sloping surface (858) may have arrows (860) formed thereon to guide a user to remove a needle cover (63) using the needle cover handling device/telescoping member (800). These outwardly sloping surfaces (858) are much larger, and therefore easier to handle than the smaller needle cover (63), thereby making the needle cover (63) removal easier using the outwardly sloping surfaces (8580 of the telescoping member (800) as well. Since the plurality of inwardly extending members (856) limit the travel of the needle cover to be along the axis of the needle (622), the needle is better protected from being bent or damaged. Thus, this also makes it easier for the user to remove the needle cover, as they are less constrained in controlling the direction of the needle cover removal, since the device controls direction for them. Also, the needle is surrounded by the movable member (850) while the needle cover is being removed. This makes the needle cover removal safer and easier because the user's hands are protected against contact with the needle during removal and protects the needle from being bent or damaged.

FIG. 14 shows the telescoping member (800) in the extended configuration. The plurality of inwardly extending members (856) has pushed the needle cover (63) distally off of the needle hub (610). At the same time, the elastic material (632) of the needle cover (63) is moved away from the sharp distal end (622) such that the distal end (622) is no longer closed by the elastic material (632). As such, any excess pressure built up in the injection system body (34) can be vented through the needle (620). Holding the injection system (100) in a vertical position during venting avoids loss of injectable fluid while allowing gas and pressure to vent.

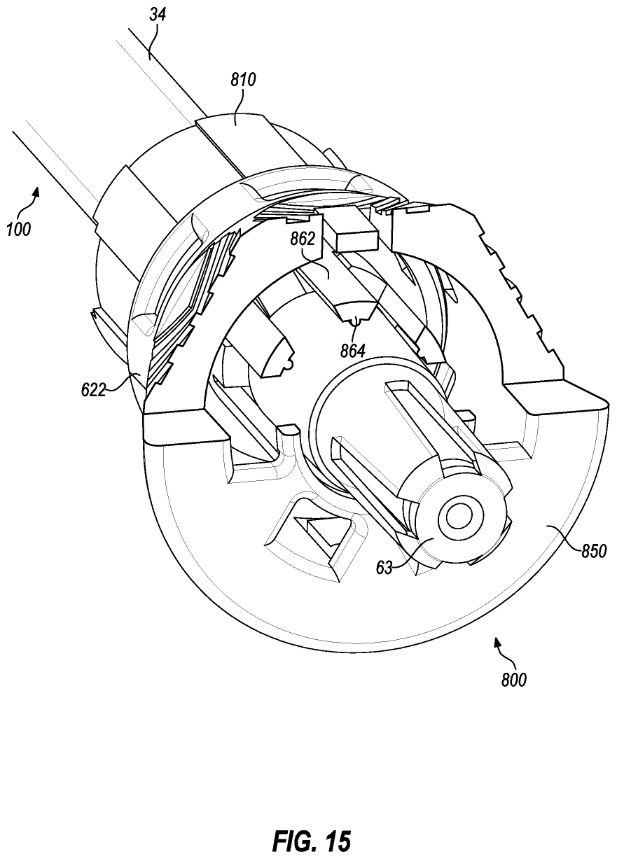

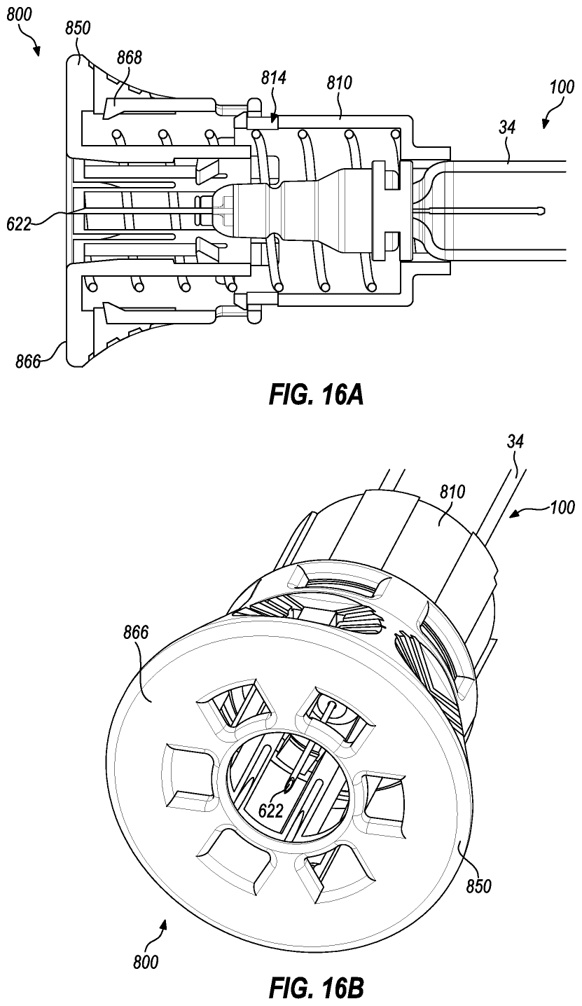

FIG. 15 shows a plurality of distally extending members (862) defined by the movable member (850). Each of the distally extending members (862) defines an inwardly extending crush rib (864) configured to removably hold the needle cover (63) when the telescoping member (800) is in the extended configuration as shown in FIG. 15. After optional venting, the user can gently pull the needle cover (63) from the crush ribs (864) of the movable member (850) to remove the needle cover (63) from the injection system (100) to prepare for an injection.

FIGS. 16A and 16B depict the injection system (100) ready for injection after removal of the needle cover (63) with the telescoping member (800) in the extended configuration. As mentioned herein, the spring (892) does not exert a resistive force noticeable to a user performing an injection using the injection system (100) with the telescoping member (800) in the extended configuration.

The distal end of the movable member (850), which forms a substantially flat distal surface (866). In the extended configuration, the distal surface (866) of the movable member (850) is substantially aligned with the distal end (622) of the needle (620) along a longitudinal axis of the injection system (100). As such, the sharp distal end (622) of the needle (620) is not visible from proximal of the movable member (850). This reduces anxiety in a patient receiving an injection by obscuring the needle (620) and the sharp distal end (622) thereof from the patient's vision. Further, the distal surface (866) of the movable member (850) makes contact completely around the injection site at the same time as or shortly before the sharp distal end (622) of the needle (620). As such, the contact around the injection site reduces/obscures the sense of pain from the injection with the sharp distal end (622) of the needle (620) by providing different tactile sensations to the nerves around the injection site. The injection angle of the injection system (100) with the telescoping member (800) installed may be optimally restricted to substantially orthogonal. Substantially orthogonal includes a maximum of about 15° from orthogonal.

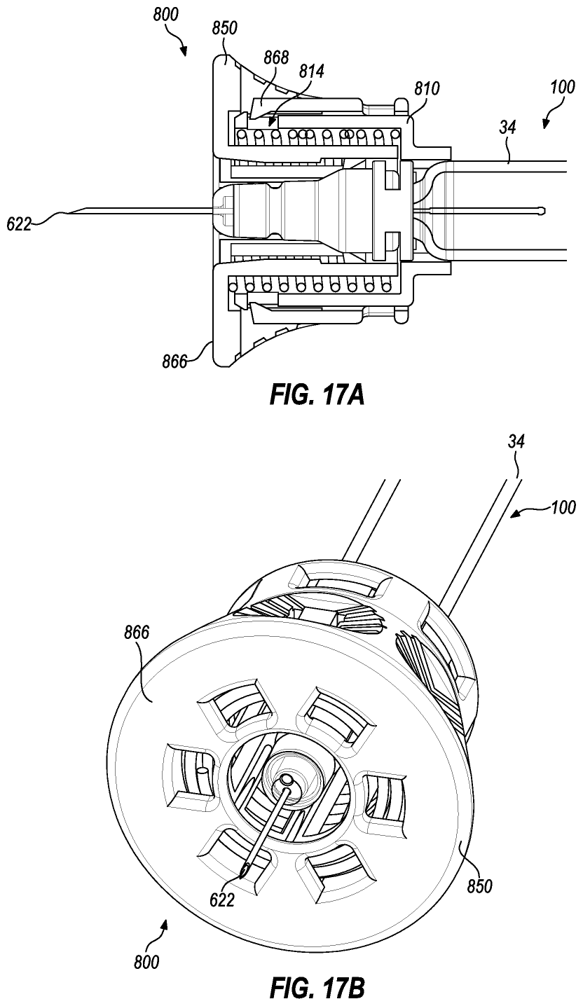

FIG. 17A and 17B depict the telescoping member (800) after injection using the injection system (100) to which the telescoping member (800) is mounted. During injection, the movable member (850) is pushed proximally relative to the fixed member (810) until the telescoping member (800) is in the fully retracted configuration depicted in FIGS. 17A and 17B. The fixed member (810) defines a plurality of side openings (814), and the movable member (850) defines a corresponding plurality of latches (868). With the telescoping member (800) in the fully retracted configuration, the latches (868) on the movable member (850) extend into the side openings (814) in the fixed member (810) and restrict the movable member (850) from moving distally relative to the fixed member (810). As such, the telescoping member (800) in the fully retracted configuration is said to be “locked out” in that it can no longer telescope to change its length. When the telescoping member (800) is “locked” in the fully retracted configuration, the combined injection system (100) and telescoping member (800) has a smaller disposal waste space footprint. This allows more used injection devices to be disposed in a “sharps” container of a particular volume.

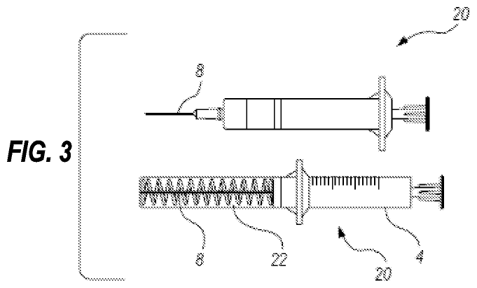

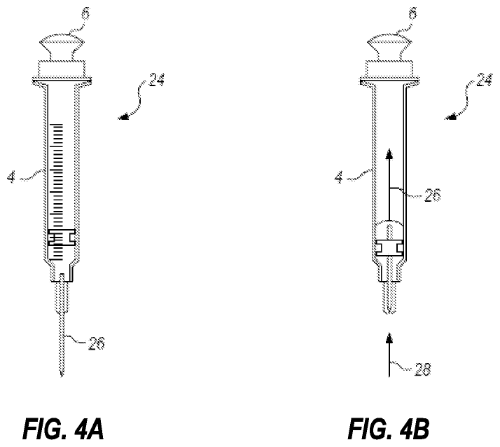

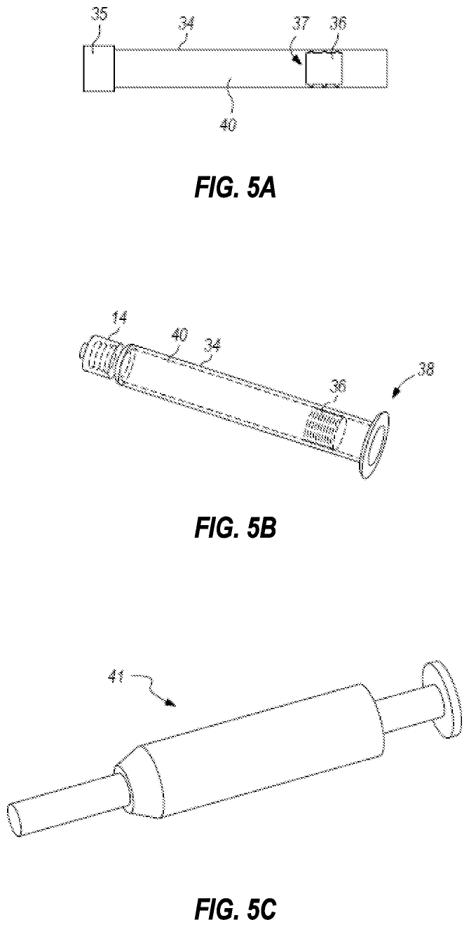

The extended configuration (FIGS. 14, 16A, and 16B) of the telescoping member (800) is different from the extended configuration of expanding needle shields, such as the tubular shield member (22) depicted in FIG. 3. In order to prevent accidental needle sticks after injection, expanding needle shields (22) are released from a proximal/retracted configuration and “locked” into a distal/extended configuration, such that the expanding needle shield (22) can no longer be returned to the proximal/retracted position to cover a needle (8) after injection. As such, the expanding needle shields (22) do not obscure a patient's view of the needle piercing the skin or reduce/obscure the sense of pain from the injection with the sharp distal end of the needle (22) by providing different tactile sensations to the nerves around the injection site. On the other hand, the telescoping member (800) expands to the extended configuration (FIGS. 14, 16A, and 16B) before injection and is “unlocked” while in the extended configuration to allow injection. As such, the telescoping member (800) both obscures a patient's view of the needle (620) piercing the skin and reduces/obscures the sense of pain from the injection with the sharp distal end (622) of the needle (620) by providing different tactile sensations to the nerves around the injection site. After injection, the telescoping member (800) is “locked” in the fully retracted configuration (FIGS. 17A and 17B) in which the distal end (622) of the needle (620) is exposed.

FIG. 17A and 17B show that the telescoping member (800) does not cover the distal end (622) of the needle (620) after injection. However, the injection system (100) may implement a needle retraction system such as that shown in FIGS. 6A to 7P to provide a safe injection system along with the needle cover management device/telescoping member (800). While the needle cover handling device/telescoping member (800) is described as facilitating removal of the needle cover (63) from the needle hub (610) utilizing distally directed force applied to the movable member (850), a user may remove the cover (63) from the needle hub (610) by pulling the needle cover (63), which is accessible to the user even with the telescoping member (800) in the retracted configuration (see FIGS. 11 and 12). Once the user removes the needle cover (63), the movable member (850) automatically springs forward to hide distal end (622) the needle (620) from view as the removal of the needle cover (63) releases the spring (892) from its compressed state.

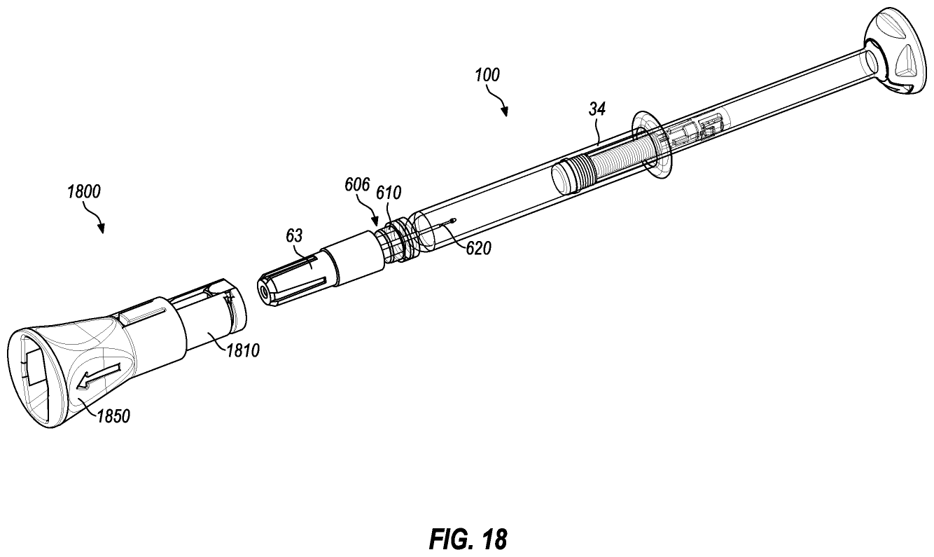

FIG. 18 depicts a needle cover handling device (1800) for use with an injection system (100) before coupling the needle cover handling device (1800) onto the injection system (100) according to some embodiments. While the injection system (100) is a single chamber injection system, the needle cover handling device (1800) can be utilized with multiple chamber injection systems such as those depicted in FIGS. 6A to 7P without any modification.

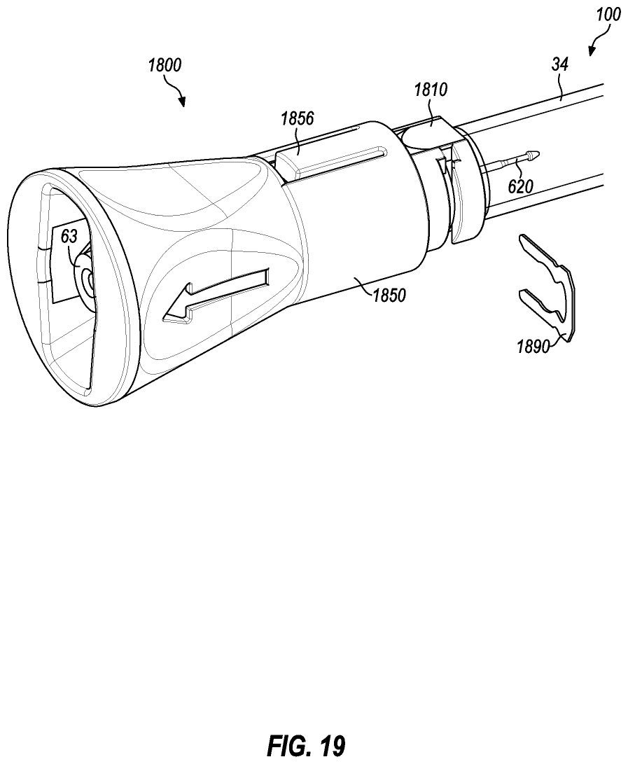

The injection system (100) includes an injection system body (34) having a needle hub assembly (606) including a needle hub (610) and a needle (620) coupled to the needle hub (610). The injection system (100) also includes a needle cover (63) (see FIG. 19) disposed over a sharp distal end (622) of the needle (620) when the injection system (100) is in a transport/storage configuration. The needle cover (63) includes an elastic material (632) configured to interfere with a circumferential groove (612) defined by the needle hub (610) to secure the needle cover (63) onto the needle hub (610). The elastic material (632) also protects the needle (620) from mechanical damage, and seals the injection system (100) before use. The needle cover (63) minimizes accidental needle sticks during use.

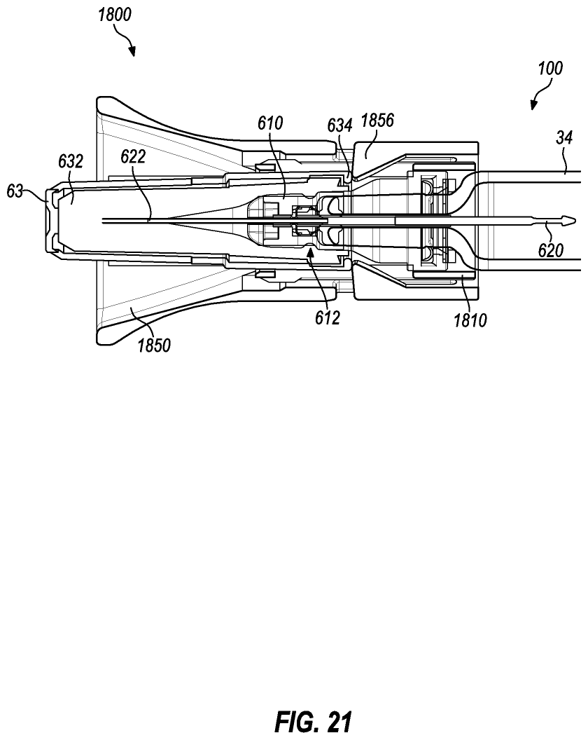

Like the needle cover handling system (800) depicted in FIGS. 8 to 17, the needle cover handling system (1800) is a telescoping sleeve (1800). The telescoping sleeve (1800) includes a fixed member (1810) and a movable member (1850), which are configured to telescope relative to each other to change the length of the telescoping sleeve (1800) (see FIG. 21). During assembly of the telescoping sleeve (1800) onto the injection system (100), the fixed member (1810) is coupled to the needle hub (610) and the injection system body (34) with a retaining clip (1890) (see FIG. 19). As seen by comparing FIGS. 18 and 19, the telescoping sleeve (1800) is installed onto the injection system (100) by sliding the telescoping sleeve (1800) over the needle cover (63) and securing the telescoping sleeve (1800) with the retaining clip (1890).

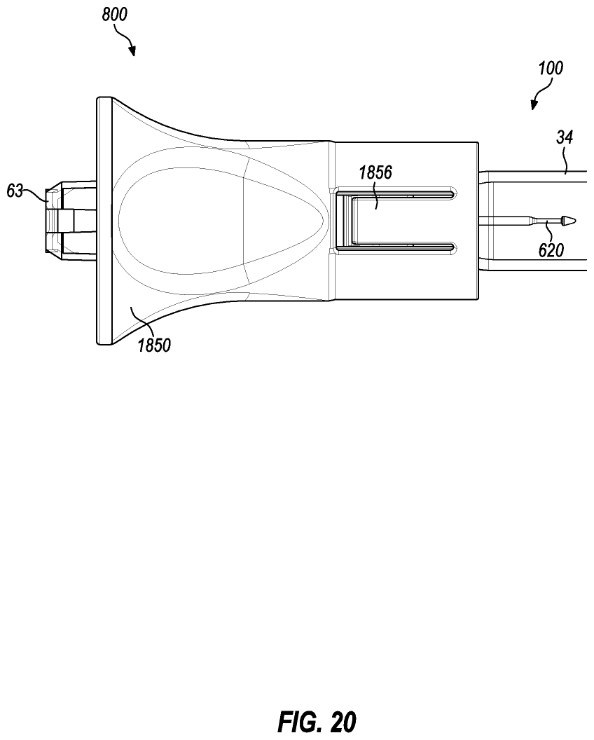

FIGS. 20 to 22 depict the telescoping sleeve (1800) installed onto the injection system (100). As shown in FIG. 21, the movable member (1850) is disposed radially outside of the fixed member (1810).

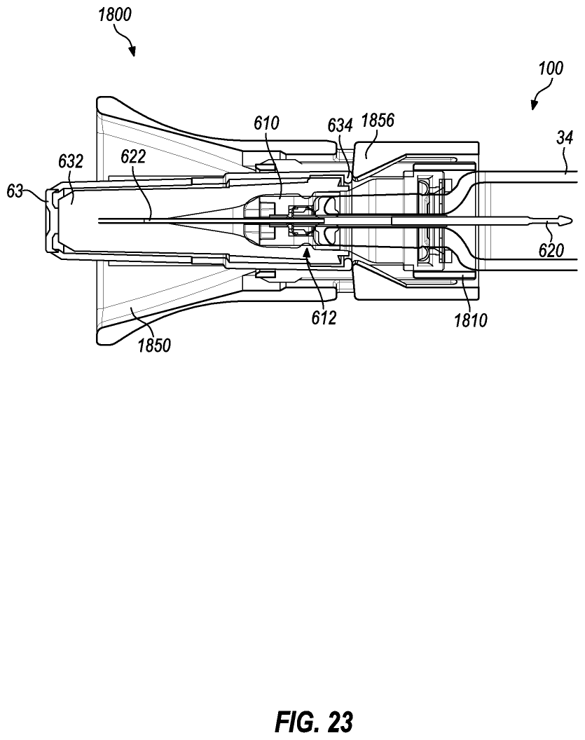

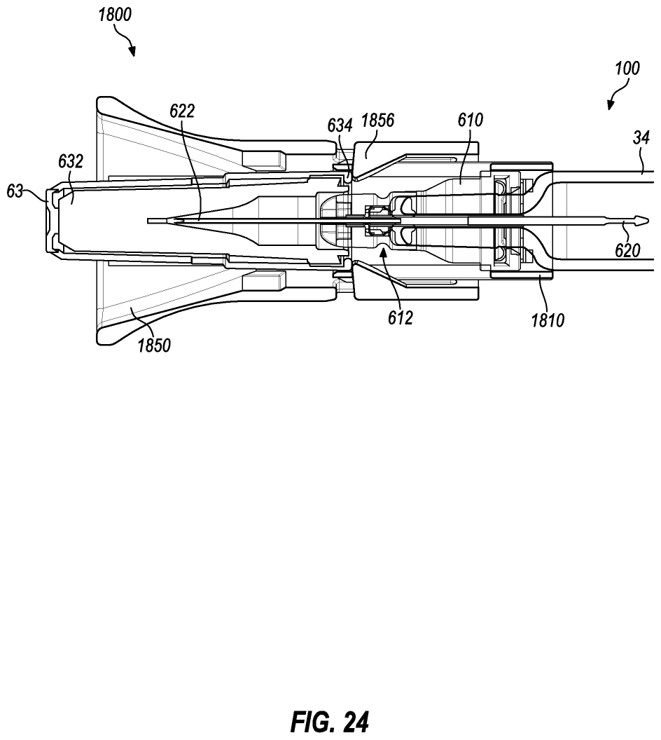

FIGS. 23 and 24 depict the telescoping member (1800) in a retracted configuration (FIG. 23) and an extended configuration (FIG. 24). In the retracted configuration depicted in FIG. 23, the movable member (1850) substantially covers the fixed member (1810). In the extended configuration depicted in FIG. 24, the movable member (1850) is moved distally relative to the fixed member (1810) relative to the retracted configuration. The telescoping member (1800) is shorter in the retracted configuration compared to the extended configuration.

The movable member (1850) also defines a pair of inwardly extending members/latches (1856) configured to interfere with a proximal end (634) of the needle cover (63) when the needle cover (63) is coupled to the needle hub (610). As such, the pair of inwardly extending members/latches (1856) are configured to exert a distally directed force from the movable member (1850) to the proximal end (634) of the needle cover (63), thereby removing the needle cover (63) from the needle hub (610). The distally directed force transferred from the movable member (1850) through the pair of inwardly extending members/latches (1856) is sufficient to overcome the resistive force between the elastic material (632) of the needle cover (63) and the outside surface of the needle hub (620) including the groove (612) form therein.

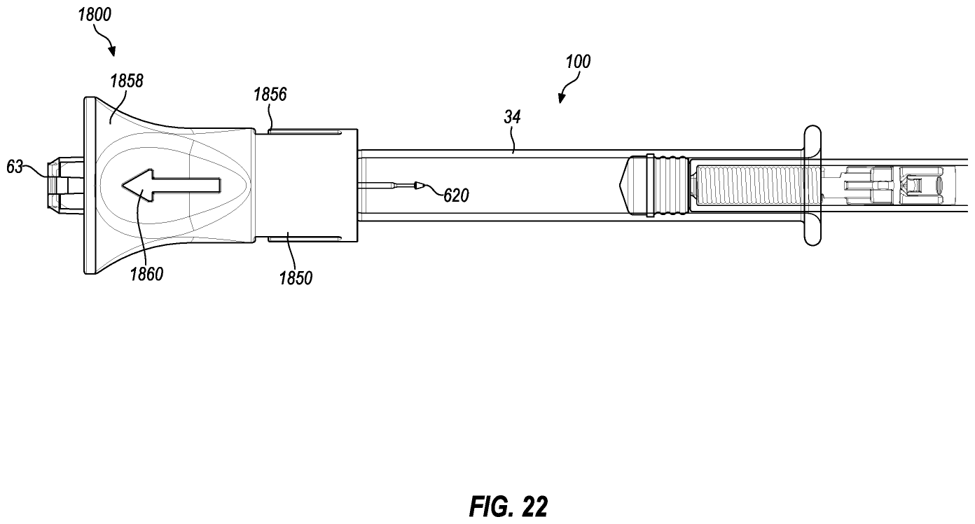

The movable member (1850) also defines an outwardly sloping surface (1858) configured to facilitate application of a distally directed force to the movable member (1850) using the fingers and thumb of one hand of a user. As shown in FIG. 22, the outwardly sloping surface (1858) may have arrows (1860) formed thereon to guide a user to remove a needle cover (63) using the needle cover handling device/telescoping member (1800). These outwardly sloping surfaces (1858) are much larger, and therefore easier to handle than the smaller needle cover (63), thereby making the needle cover (63) removal using the outwardly sloping surfaces (1858) of the telescoping member (1800) easier as well. Since the plurality of inwardly extending members (856) limit the travel of the needle cover to be along the axis of the needle (622), the needle is better protected from being bent or damaged. Thus, this also makes it easier for the user to remove the needle cover (63), as the user is less constrained in controlling the direction of the needle cover (63) removal, since the needle cover management device/telescoping member (1800) controls the direction of needle (63) removal. Also, the distal end (622) of the needle (620) is surrounded by the movable member (850) while the needle cover (63) is being removed. This makes the needle cover (63) removal safer and easier because the user's hands are protected against contact with the needle (620) during removal and protects the needle (620) from being bent or damaged.

FIG. 24 shows the telescoping member (1800) in the extended configuration. The pair of inwardly extending members/latches (1856) has pushed the needle cover (63) distally off of the needle hub (610). At the same time, the elastic material (632) of the needle cover (63) is moved away from the sharp distal end (622) such that the distal end (622) is no longer closed by the elastic material (632). As such, any excess pressure built up in the injection system body (34) can be vented through the needle (620). Holding the injection system (100) in a vertical position during venting avoids loss of injectable fluid while allowing gas and pressure to vent.

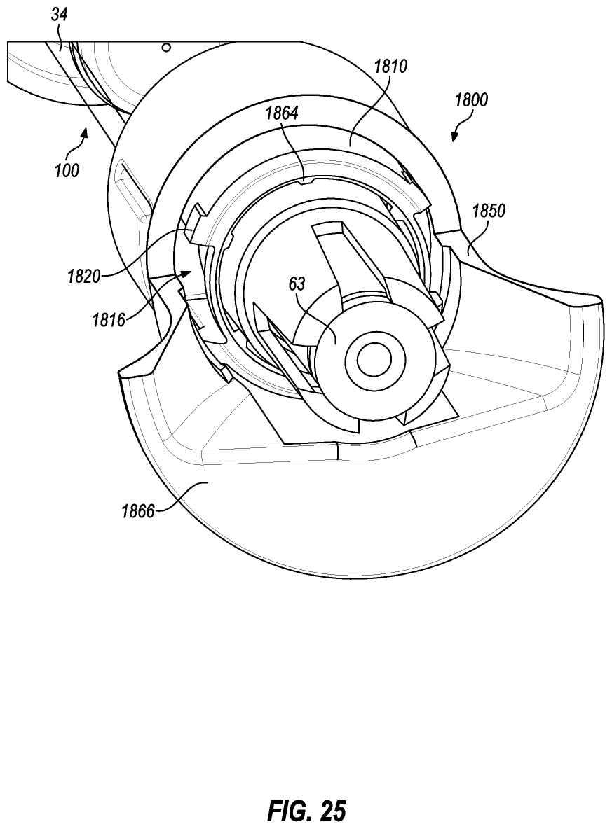

FIG. 25 shows a plurality of inwardly extending skids (1864) defined by the fixed member (1810). The plurality of inwardly extending skids (1864) are configured to removably hold the needle cover (63) when the telescoping member (1800) is in the extended configuration as shown in FIGS. 24 and 25. After optional venting, the user can gently pull the needle cover (63) from the inwardly extending skids (1864) of the movable member (1850) to remove the needle cover (63) from the injection system (100) to prepare for an injection.

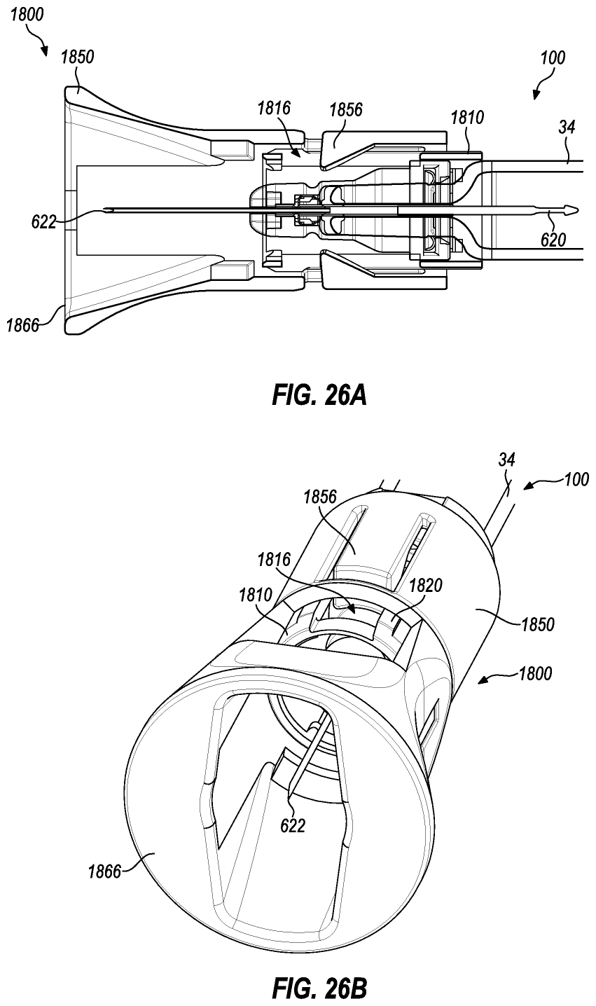

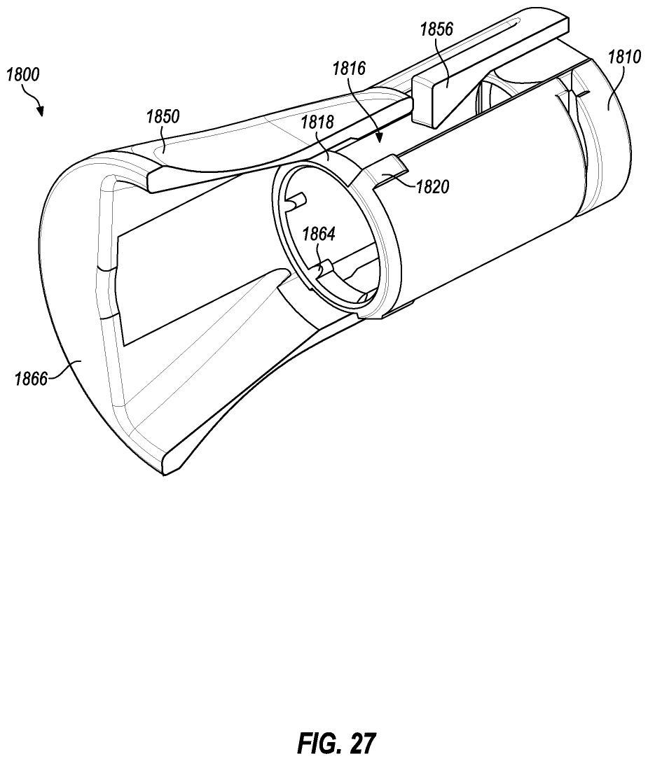

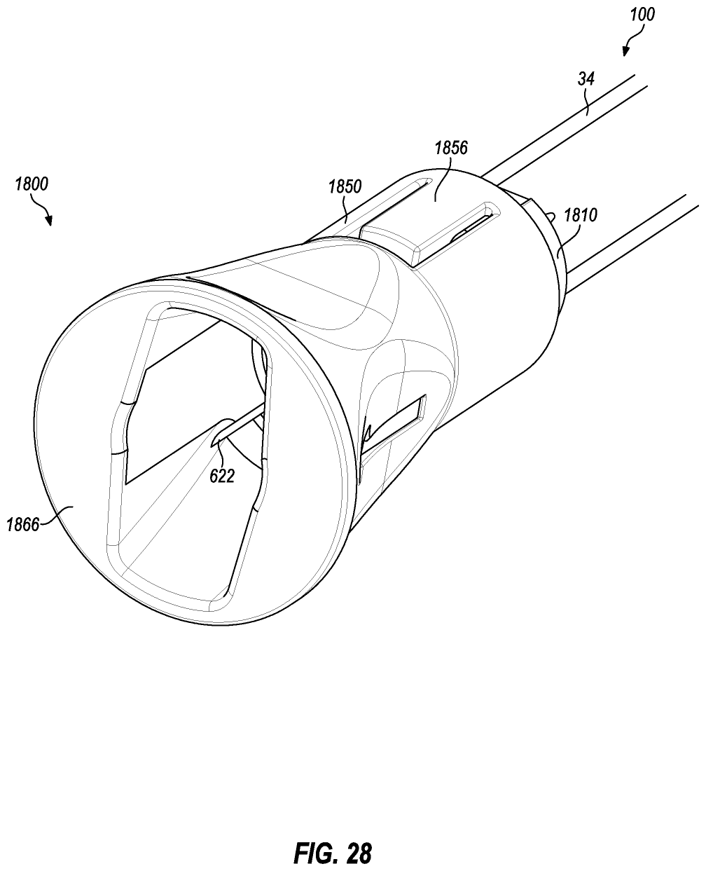

FIGS. 26A, 26B, 27, and 28 depict the injection system (100) ready for injection after removal of the needle cover (63) with the telescoping member (1800) in the extended configuration. The fixed member (1810) defines a pair of longitudinal slots (1816) extending to a distal end thereof, and corresponding to the pair of inwardly extending members/latches (1856) defined by the movable member (1850). The pair of inwardly extending members/latches (1856) are configured to interfere with proximally facing surfaces (1818) of the fixed member (1810) defining a distal end of respective longitudinal slots (1816) to limit distal movement of the movable member (1850) relative to the fixed member (1810), as shown in FIG. 26A, 26B, and 27. A plurality of ramps (1820) outwardly extending from the fixed member (1810) increase the frictional fit between the fixed and movable members (1810, 1850) when the telescoping member (1800) is in the extended configuration. Any resistive frictional force between the fixed and movable members (1810, 1850) is not noticeable to a user performing an injection using the injection system (100) with the telescoping member (1800) in the extended configuration.

The distal end of the movable member (1850), which forms a substantially flat distal surface (1866). In the extended configuration, the distal surface (1866) of the movable member (1850) is distal of the distal end (622) of the needle (620) along a longitudinal axis of the injection system (100). As such, the sharp distal end (622) of the needle (620) is not visible from proximal of the movable member (1850). This reduces anxiety in a patient receiving an injection by obscuring the needle (620) and the sharp distal end (622) thereof from the patient's vision. Further, the distal surface (1866) of the movable member (1850) makes contact completely around the injection site shortly before the sharp distal end (622) of the needle (620). As such, the contact around the injection site reduces/obscures the sense of pain from the injection with the sharp distal end (622) of the needle (620) by providing different tactile sensations to the nerves around the injection site. The injection angle of the injection system (100) with the telescoping member (1800) installed may be optimally restricted to substantially orthogonal. Substantially orthogonal includes a maximum of 15° to 30° from orthogonal.

While the needle cover handling device/telescoping member (1800) is described as facilitating removal of the needle cover (63) from the needle hub (610) utilizing distally directed force applied to the movable member (1800), a user may remove the cover (63) from the needle hub (610) by pulling the needle cover (63), which is accessible to the user with the telescoping member (1850) in the retracted configuration (see FIG. 23). Even though the user removes the needle cover (63), the movable member (1850) still covers the distal end (622) of the needle (620), thereby protecting the user's hand if they choose to remove the needle cover (63) by pulling thereon.

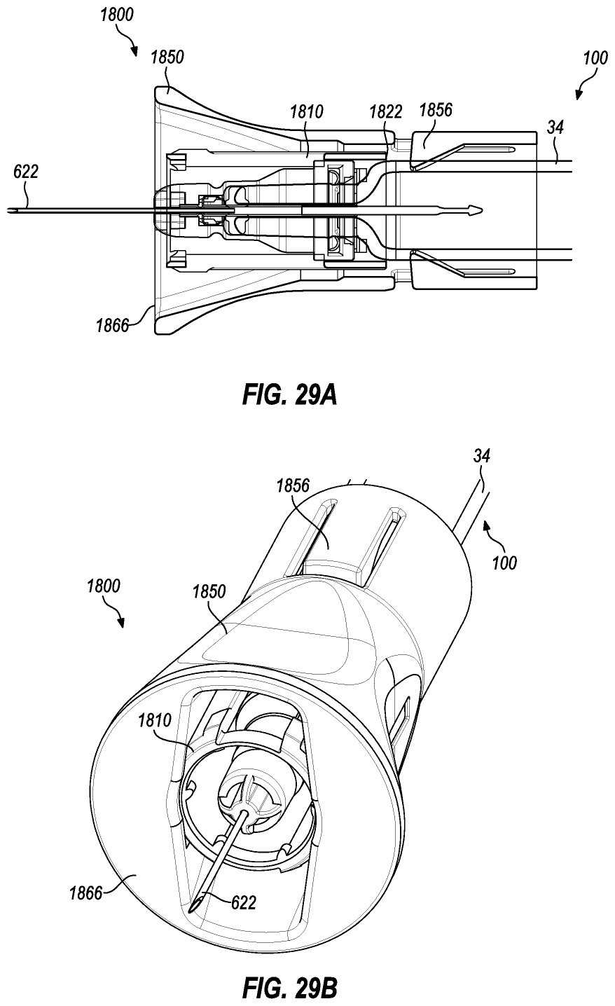

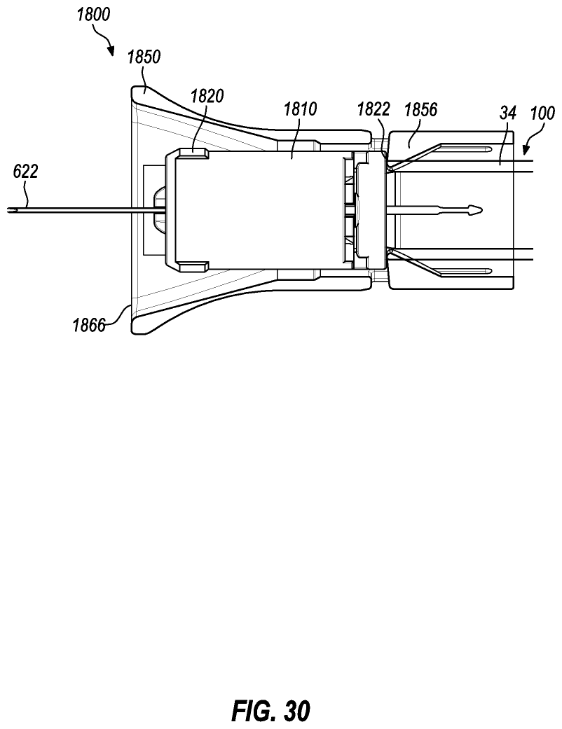

FIG. 29A, 29B, and 30 depict the telescoping member (1800) after injection using the injection system (100) to which the telescoping member (1800) is mounted. During injection, the movable member (1850) is pushed proximally relative to the fixed member (1810) until the telescoping member (1800) is in the fully retracted configuration depicted in FIGS. 29A, 29B, and 30. With the telescoping member (1800) in the fully retracted configuration, the pair of inwardly extending members/latches (1856) defined by the movable member (1850) have moved proximally out of the pair of longitudinal slots (1816) (guided by the ramp built into each inwardly extending member/latch (1856). The pair of inwardly extending members/latches (1856) interfere with a proximally facing end surface (1822) of the fixed member (1810), and restrict the movable member (1850) from moving distally relative to the fixed member (1810). As such, the telescoping member (1800) in the fully retracted configuration is said to be “locked out” in that it can no longer telescope to change its length. When the telescoping member (1800) is “locked” in the fully retracted configuration, the combined injection system (100) and telescoping member (1800) has a smaller disposal waste space footprint. This allows more used injection devices to be disposed in a “sharps” container of a particular volume.

The extended configuration (FIGS. 26A, 26B, 27, and 28) of the telescoping member (1800) is different from the extended configuration of expanding needle shields, such as the tubular shield member (22) depicted in FIG. 3. In order to prevent accidental needle sticks after injection, expanding needle shields (22) are released from a proximal/retracted configuration and “locked” into a distal/extended configuration, such that the expanding needle shield (22) can no longer be returned to the proximal/retracted position to cover a needle (8) after injection. As such, the expanding needle shields (22) do not obscure a patient's view of the needle piercing the skin or reduce/obscure the sense of pain from the injection with the sharp distal end of the needle (22) by providing different tactile sensations to the nerves around the injection site. On the other hand, the telescoping member (1800) expands to the extended configuration (FIGS. 26A, 26B, 27, and 28) before injection and is “unlocked” while in the extended configuration to allow injection. As such, the telescoping member (1800) both obscures a patient's view of the needle (620) piercing the skin and reduces/obscures the sense of pain from the injection with the sharp distal end (622) of the needle (620) by providing different tactile sensations to the nerves around the injection site. After injection, the telescoping member (1800) is “locked” in the fully retracted configuration (FIGS. 29A, 29B, and 30) in which the distal end (622) of the needle (620) is exposed.

FIG. 29A, 29B, and 30 show that the telescoping member (1800) does not cover the distal end (622) of the needle (620) after injection. However, the injection system (100) may implement a needle retraction system such as that shown in FIGS. 6A to 7P to provide a safe injection system along with the needle cover management device/telescoping member (1800). While the needle cover handling device/telescoping member (1800) is described as facilitating removal of the needle cover (63) from the needle hub (610) utilizing distally directed force applied to the movable member (1850), a user may remove the cover (63) from the needle hub (610) by pulling the needle cover (63), which is accessible to the user even with the telescoping member (1800) in the retracted configuration (see FIGS. 20 and 21). However, most of the access to the needle cover (63) is blocked by the movable member (1850) when the telescoping member (1800) in the retracted configuration, thereby discouraging users from pulling off the needle cover (63) and encouraging users to use the telescoping member (1800) to remove the needle cover (63). Although users may remove the needle cover (63) manually using their hands, when the movable member (1850) is in the retracted condition (see FIGS. 20 and 21), the movable member (1800) still covers the distal end (622) of the needle (620).

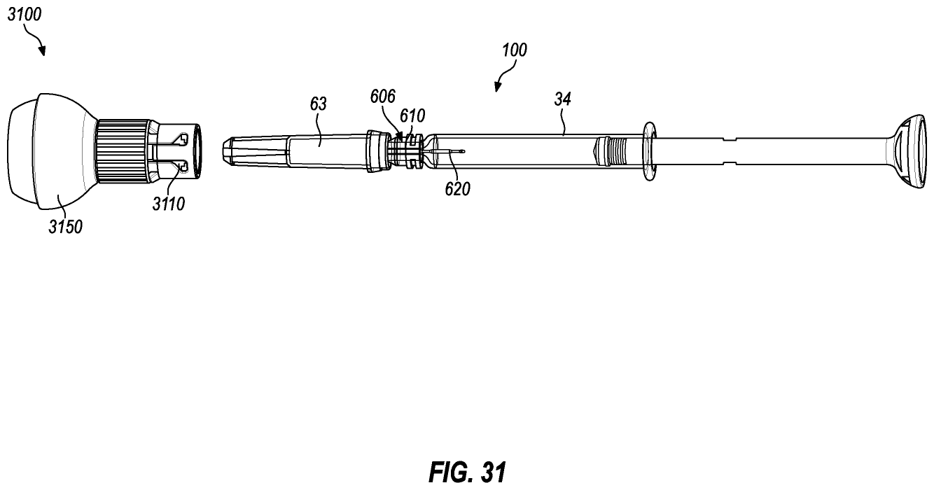

FIG. 31 depicts a needle cover handling device (3100) for use with an injection system (100) before coupling the needle cover handling device (3100) onto the injection system (100) according to some embodiments. While the injection system (100) is a single chamber injection system, the needle cover handling device (3100) can be utilized with multiple chamber injection systems such as those depicted in FIGS. 6A to 7P without any modification.

The injection system (100) includes an injection system body (34) having a needle hub assembly (606) including a needle hub (610) and a needle (620) coupled to the needle hub (610). The injection system (100) also includes a needle cover (63) disposed over a sharp distal end of the needle (620) when the injection system (100) is in a transport/storage configuration. The needle cover (63) minimizes accidental needle sticks during use.

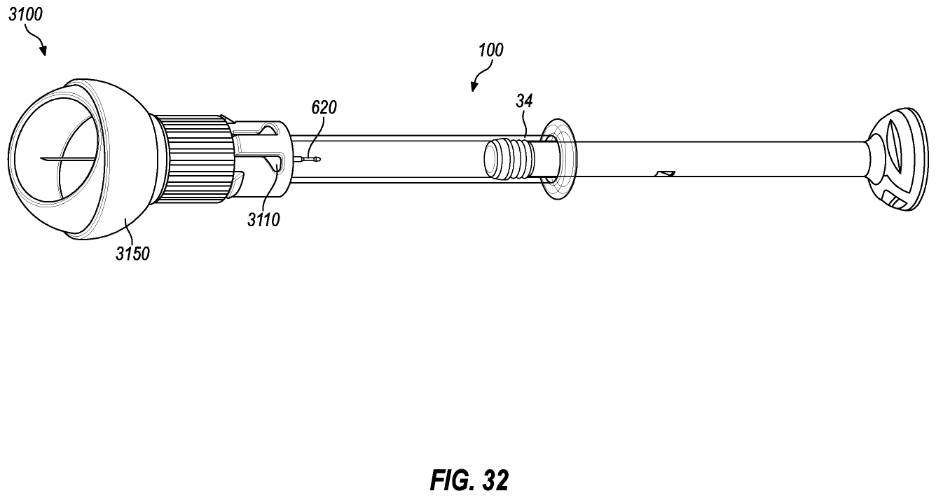

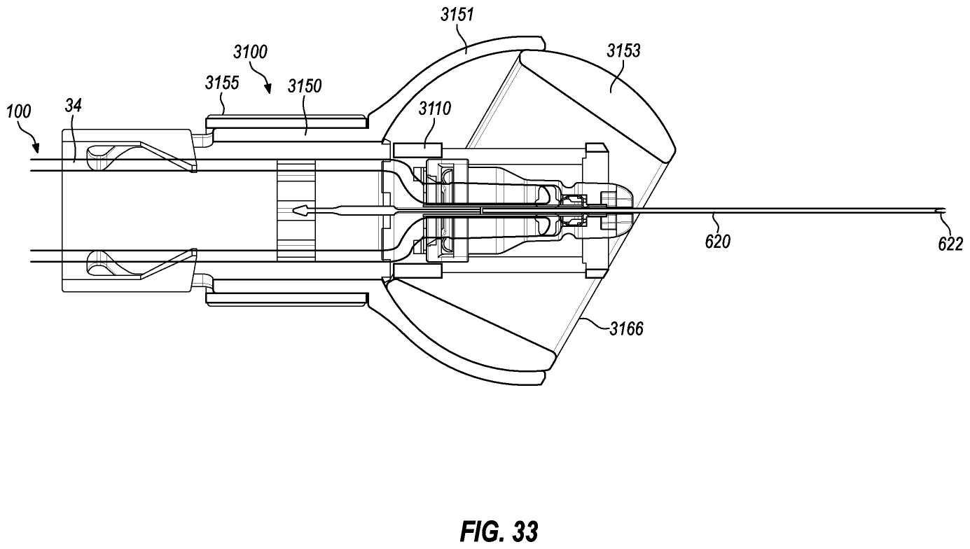

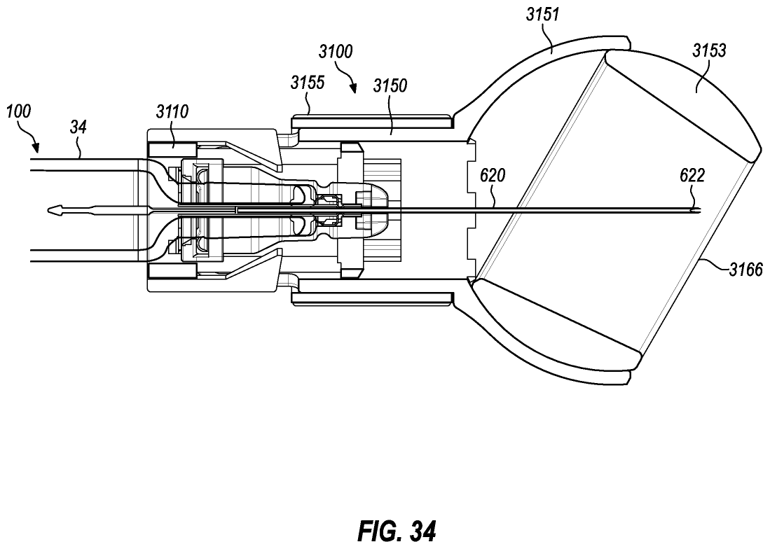

Like the needle cover handling systems (800, 1800) depicted in FIGS. 8 to 17 and 18 to 30, the needle cover handling system (3100) is a telescoping sleeve (3100). The telescoping sleeve (3100) includes a fixed member (3110) and a movable member (3150), which are configured to telescope relative to each other to change the length of the telescoping sleeve (3100) (see FIGS. 33 and 34). As seen by comparing FIGS. 31 and 32, the telescoping sleeve (3100) is installed onto the injection system (100) by sliding the telescoping sleeve (3100) over the needle cover (63) and securing the telescoping sleeve (3100) with the retaining clip (not shown).

FIGS. 33 and 34 depict the telescoping member (3100) in a retracted configuration (FIG. 33) and an extended configuration (FIG. 34). In the retracted configuration depicted in FIG. 33, the movable member (3150) substantially covers a middle portion of the fixed member (3110). In the extended configuration depicted in FIG. 34, the movable member (3150) is moved distally relative to the fixed member (3110) relative to the retracted configuration. The telescoping member (3100) is shorter in the retracted configuration compared to the extended configuration.

FIGS. 33 and 34 also show that a distal end of the movable member (3150) defines a dome (3151) in which a swivel cup (3153) is disposed. The swivel cup facilitates the injection system (100) achieving injection angles of up to 30° from orthogonal, and even up to 45° from orthogonal when the telescoping member (3100) is installed thereon.

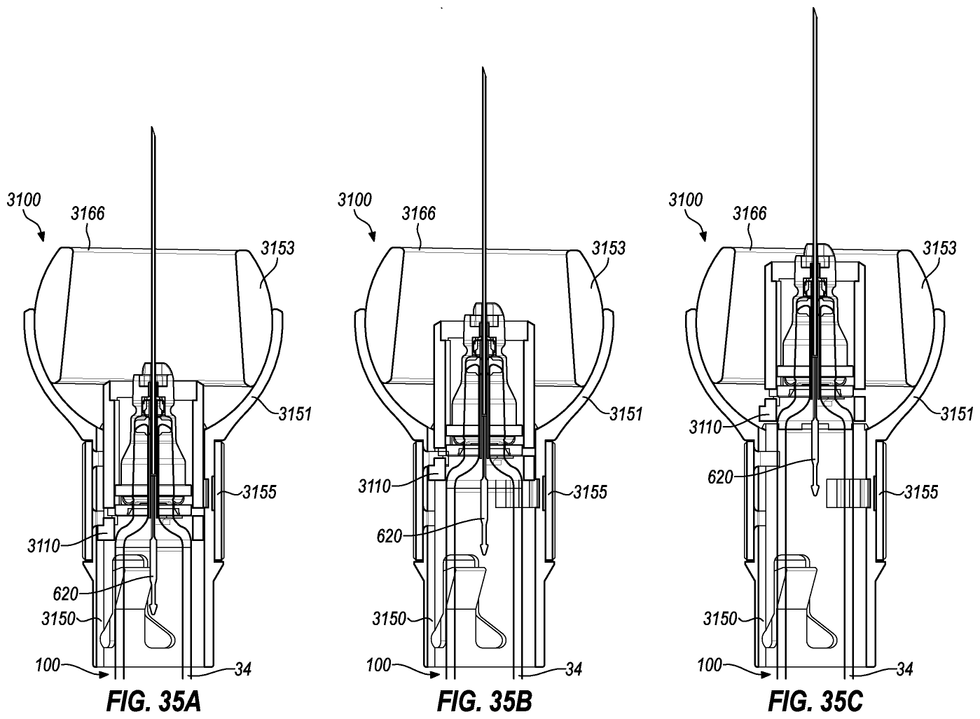

FIGS. 33 and 34 also show that the movable member (3150) includes an adjustment dial (3155) configured to allow a user to set a needle length. FIGS. 35A, 35B, and 35C depict a needle cover handling device/telescoping member (3100) mounted on an injection system (100) with the telescoping member (3100) “locked” in respective short, medium, and long fully retracted configurations according to some embodiments. Rotating the adjustment dial (3155) adjusts a relative position of the movable member (3150) and the fixed member (3110) to adjusts a needle length both before or after a needle cover has been removed. For instance, FIG. 35A depicts a needle length of 0.5″, FIG. 35B depicts a needle length of 0.75″, and FIG. 35C depicts a needle length of 1.0″.

When the telescoping member (3100) is “locked” in the fully retracted configuration, the combined injection system (100) and telescoping member (3100) has a smaller disposal waste space footprint. This allows more used injection devices to be disposed in a “sharps” container of a particular volume.

As shown in FIG. 34, the distal end of the swivel cup (3153) of the movable member (3150), forms a substantially flat distal surface (3166). In the extended configuration shown in FIG. 34, the distal surface (3166) of the swivel cup (3153) of the movable member (3150) is substantially aligned with the distal end (622) of the needle (620) along a longitudinal axis of the injection system (100). As such, the sharp distal end (622) of the needle (620) is not visible from proximal of the movable member (3150). This reduces anxiety in a patient receiving an injection by obscuring the needle (620) and the sharp distal end (622) thereof from the patient's vision. Further, the distal surface (3166) of the swivel cup (3153) of the movable member (3150) makes contact completely around the injection site at the same time as or shortly before the sharp distal end (622) of the needle (620). As such, the contact around the injection site reduces/obscures the sense of pain from the injection with the sharp distal end (622) of the needle (620) by providing different tactile sensations to the nerves around the injection site.

The extended configuration (FIG. 34) of the telescoping member (3100) is different from the extended configuration of expanding needle shields, such as the tubular shield member (22) depicted in FIG. 3. In order to prevent accidental needle sticks after injection, expanding needle shields (22) are released from a proximal/retracted configuration and “locked” into a distal/extended configuration, such that the expanding needle shield (22) can no longer be returned to the proximal/retracted position to cover a needle (8) after injection. As such, the expanding needle shields (22) do not obscure a patient's view of the needle piercing the skin or reduce/obscure the sense of pain from the injection with the sharp distal end of the needle (22) by providing different tactile sensations to the nerves around the injection site. On the other hand, the telescoping member (3100) expands to the extended configuration (FIG. 34) before injection and is “unlocked” while in the extended configuration to allow injection. As such, the telescoping member (3100) both obscures a patient's view of the needle (620) piercing the skin and reduces/obscures the sense of pain from the injection with the sharp distal end (622) of the needle (620) by providing different tactile sensations to the nerves around the injection site. After injection, the telescoping member (3100) is “locked” in the fully retracted configuration (FIGS. 35A, 35B, and 35C) in which the distal end (622) of the needle (620) is exposed to different extents depending on user selection of needle length by rotation of the adjustment dial (3155).

FIG. 35A, 35B, and 35C show that the telescoping member (3100) does not cover the distal end (622) of the needle (620) after injection. However, the injection system (100) may implement a needle retraction system such as that shown in FIGS. 6A to 7P to provide a safe injection system along with the needle cover management device/telescoping member (3100).

While the embodiments described above include single and dual chamber (safety) injection systems, the scope of the claims also include other multiple chamber injection systems, with or without safe injection systems. For multiple chamber safety injection systems with more than two chambers, more than two stopper members are inserted into an injection system body (e.g., syringe body, cartridge body, etc.) to define a corresponding number of chambers.

While the injection systems depicted and described herein include syringes with staked needles, the needle cover handling devices/telescoping members described herein can be used with cartridges, an auto injector, and injection systems with Luer connectors, etc.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, ETFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed is:

1. A system for injection, comprising:
a syringe body having proximal and distal ends and defining a syringe interior;
a stopper member disposed in the syringe interior;
a plunger member coupled to the stopper member and configured to be manipulated to insert the stopper member distally in the syringe interior relative to the syringe body;
a needle hub assembly coupled to the syringe body at the distal end thereof and comprising
a needle hub, and
a needle coupled to the needle hub;
a needle shield removably coupled to the needle hub over the needle; and
a telescoping sleeve coupled to the distal end of the syringe body at least partially over the needle shield.

2. The system of claim 1, wherein the needle hub defines a circumferential groove,
wherein the needle shield comprises an elastic material configured to interfere with the circumferential groove in the needle hub to resist removal of the needle shield from the needle hub.

3. The system of claim 1, wherein the telescoping sleeve comprises fixed and movable members configured to telescope relative to each other along a longitudinal axis of the telescoping sleeve.

4. The system of claim 3, wherein the movable member is disposed at least partially radially outside of the fixed member.

5. The system of claim 3, wherein the movable member defines an annular space, and
wherein the fixed member is at least partially disposed in the annular space.

6. The system of claim 5, wherein the telescoping sleeve comprises a spring disposed in the annular space radially inward of the fixed member, and
wherein the spring reduces a force required to remove the needle shield from the needle hub.

7. The system of claim 6, wherein the spring does not exert a resistive force noticeable to a user performing an injection using the system.

8. The system of claim 3, wherein the movable member defines a plurality of inwardly extending members configured to interfere with a proximal end of the needle shield when the needle shield is coupled to the needle hub.

9. The system of claim 8, wherein the plurality of inwardly extending members are configured to exert a distally directed force from the movable member to the proximal end of the needle shield when the needle shield is coupled to the needle hub.

10. The system of claim 3, wherein the fixed member defines an outwardly extending flange at a distal end thereof,
wherein the movable member defines an inwardly extending flange at a proximal end thereof, and
wherein the outwardly extending flange and the inwardly extending flange are configured to interfere with each other to limit distal movement of the movable member relative to the fixed member.

11. The system of claim 3, wherein the movable member defines an outwardly sloping surface configured to facilitate application of a distally directed force to the movable member of the telescoping sleeve.

12. The system of claim 3, wherein the telescoping sleeve has a retracted configuration and an extended configuration,
wherein the fixed and movable members substantially overlap each other when the telescoping sleeve is in the retracted configuration, and
wherein the movable member is moved distally relative to the fixed member with minimal overlap between the fixed and movable members when the telescoping sleeve is in the extended configuration.

13. The system of claim 12, wherein the telescoping sleeve also has a fully retracted configuration,
wherein the fixed member defines a side opening,
wherein the movable member comprises a latch configured to enter the opening when the telescoping sleeve is in the fully retracted configuration, thereby preventing the movable member from moving distally relative to the fixed member.

14. The system of claim 12, wherein a distal end of the movable member is substantially aligned with a distal tip of the needle along a longitudinal axis of the system when the telescoping sleeve is in the extended configuration.

15. The system of claim 12, wherein a distal end of the movable member prevents a distal tip of the needle from being seen from proximal of the movable member when the telescoping sleeve is in the extended configuration.

16. The system of claim 12, wherein a distal end of the movable member is configured to contact a patient's skin at substantially the same time as a distal tip of the needle during an injection using the system when the telescoping sleeve is in the extended configuration.

17. The system of claim 1, further comprising a retaining clip configured to secure the telescoping sleeve to the needle hub.

18. The system of claim 1, wherein the movable member defines a plurality of distally extending members configured to removably retain the needle shield away from a distal end of the needle after the needle shield is removed from the needle hub.

19. The system of claim 18, wherein each of the plurality of distally extending members defines an inwardly extending rib configured to contact the needle shield after the needle shield is removed from the needle hub.

20. The system of claim 1, wherein the movable member defines a substantially flat distally facing surface.

21. The system of claim 1, further comprising a safe rejection system configured to retract the needle at least partially within the syringe body after an injection with the system is completed such that a distal end of the needle is disposed within the needle hub or the syringe body.

22. The system of claim 3, wherein the movable member defines a pair of inwardly extending latches configured to interfere with a proximal end of the needle shield when the needle shield is coupled to the needle hub.

23. The system of claim 22, wherein the pair of inwardly extending latches are configured to exert a distally directed force from the movable member to the proximal end of the needle shield when the needle shield is coupled to the needle hub.

24. The system of claim 22, wherein the telescoping sleeve has a retracted configuration and an extended configuration,
wherein the movable member substantially covers the fixed member when the telescoping sleeve is in the retracted configuration, and
wherein the movable member is moved distally relative to the fixed member when the telescoping sleeve is in the extended configuration.

25. The system of claim 24, wherein the fixed member defines a pair of slots,
wherein the pair of inwardly extending latches are partially disposed in the pair of slots when the telescoping member is in the retracted or extended configurations, and
wherein the pair of inwardly extending latches are configured to interfere with respective proximally facing surfaces at the distal ends of the pair of slots to limit distal movement of the movable member relative to the fixed member.

26. The system of claim 24, wherein the fixed member defines a plurality of inwardly extending skids configured to contact an inner surface of the movable member to retain the telescoping member in the extended configuration.

27. The system of claim 24, wherein the fixed member defines an inwardly extending rib configured to contact the needle shield after the needle shield is removed from the needle hub.

28. The system of claim 24, wherein the telescoping sleeve also has a fully retracted configuration,
wherein the pair of inwardly extending latches are also configured to interfere with a proximal surface of the fixed member when the telescoping sleeve is in the fully retracted configuration, thereby preventing the movable member from moving distally relative to the fixed member.

29. The system of claim 24, wherein a distal end of the movable member is substantially aligned with a distal tip of the needle along a longitudinal axis of the system when the telescoping sleeve is in the retracted configuration.
